(19) United States
(12) Patent Application Publication
(10) Pub. No.: US 2017/0232070 A1
(43) Pub. Date: Aug. 17, 2017

(54) Combination Methods for Immunotherapy

(71) Applicant: Richard P. Junghans, Boston, MA (US)

(72) Inventor: Richard P. Junghans, Boston, MA (US)

(21) Appl. No.: 15/333,127

(22) Filed: Oct. 24, 2016

Related U.S. Application Data
(60) Provisional application No. 62/245,961, filed on Oct. 23, 2015.

(51) Int. Cl. A61K 38/20; A61K 45/06; A61K 39/395; C07K 16/30; A61K 38/17; A61K 9/00; A61K 35/17

(52) U.S. Cl. CPC A61K 38/2013; A61K 9/0019; A61K 45/06; A61K 35/17; C07K 16/3069; A61K 38/1774; A61K 39/39558; A61K 2035/124

(57) ABSTRACT

The present invention includes a method of treating prostate cancer in a human subject in need thereof, comprising administering to the subject an effective amount of a composition comprising interleukin-2 (IL2), and administering to the subject a cell expressing a chimeric antigen receptor (CAR) which specifically binds prostate specific membrane antigen (PSMA), thereby treating prostate cancer in the human subject in need thereof.

### RELATED APPLICATIONS

This application claims priority to U.S. Provisional Patent Application No. 62/245,961, filed on Oct. 23, 2015, the entire contents of which are expressly incorporated herein by reference.

### BACKGROUND OF THE INVENTION

In 2012, 241,740 new cases of prostate cancer and 28,170 deaths were estimated for the US [1]. In patients with advanced disease, the 5-year survival was 29% for 2001-07 [1]. Androgen deprivation therapy (ADT) is useful for 1-3 years, recently augmented with agents abiraterone and enzalutamide [2, 3]. In patients with castrate resistant prostate cancer (CRPC), incremental benefit was obtained with chemotherapies docetaxel and cabazitaxel [4, 5]. Sipuleucel-T, an autologous “therapeutic vaccine,” adds further months of survival [6]. No treatment has yet proven curative in metastatic settings.

Accordingly, there remains a need for therapies that can be used for therapeutic purposes for treating cancer.

### SUMMARY OF THE INVENTION

The invention provides a combination therapy using IL2 therapy and designer T cells (also referred to as CAR-T cells) to treat cancer, such as prostate cancer.

The invention includes a method of treating prostate cancer in a human subject in need thereof, comprising administering to the subject an effective amount of a composition comprising interleukin-2 (IL2), and administering to the subject a cell expressing a chimeric antigen receptor (CAR) which specifically binds prostate specific membrane antigen (PSMA), thereby treating prostate cancer in the human subject in need thereof.

In one embodiment, the prostate cancer is associated with high levels of expression of PSMA.

In one embodiment, the prostate cancer is metastatic pancreatic cancer, recurrent prostate cancer or hormone-refractory prostate cancer.

In one embodiment, the method further comprises administering cyclophosphamide to the human subject.

In one embodiment, the method further comprises administering fludarabine to the human subject. In one embodiment, the fludarabine is administered to the human subject after the cyclophosphamide is administered to the human subject. In one embodiment, the cell expressing a CAR which specifically binds PSMA is administered to the human subject after the fludarabine is administered to the human subject.

In one embodiment, the composition comprising IL2 is administered to the human subject by continuous intravenous infusion at a dose of about 75000 IU/kg/d for 3 to 48 days, 7 to 44 days, 10 to 40 days, 14 to 36 days, 20 to 32 days, about 7 days, about 3 months (or 90 days), or about 28 days. Ranges intermediate to those recited are also included in the possible frequency with which IL2 is administered.

In one embodiment, the composition comprising IL2 is aldesleukin (Proleukin). In one embodiment, the human subject is administered 1×10⁹ to 1×10¹¹ cells expressing a CAR which specifically binds PSMA.

In one embodiment, the cell expressing a CAR which specifically binds PSMA has been activated with an anti-CD3 antibody prior to administration to the human subject.

In one embodiment, the CAR comprises a PSMA binding region of an anti-PSMA antibody and a CD3 zeta signaling chain of a T cell receptor.

In one embodiment, the anti-PSMA antibody is 3D8.

In one embodiment, the cell is a T-cell obtained from the subject.

In one aspect, provided herein is a method of treating prostate cancer in a human subject in need thereof, comprising administering to the subject a population of cells expressing a chimeric antigen receptor (CAR) which specifically binds prostate specific membrane antigen (PSMA) and administering interleukin-2 (IL2), thereby treating prostate cancer in the human subject, wherein the IL2 is administered to the human subject by continuous intravenous infusion at a dose of about 75000 IU/kg/d and is administered after administration of the population of cells expressing the CAR.

In one embodiment, the method further comprises administering cyclophosphamide and/or fludarabine to the human subject.

In one embodiment, the IL2 is administered to the subject for about 28 days by continuous intravenous infusion.

In one embodiment, the CAR comprises a PSMA binding region of an anti-PSMA antibody and a CD3 zeta signaling region of a T cell receptor.

In one embodiment, the anti-PSMA antibody is 3D8, or an antigen binding fragment thereof.

In another aspect, provided herein is a method of treating a human subject having prostate cancer, said method comprising administering a population of cells expressing an anti-PSMA CAR to the human subject and administering IL2 to the human subject, wherein the IL2 is administered intravenously to the human subject at a dose of 100 kIU/kg/8 h or more by bolus infusion and is administered after administration of the population of cells expressing the anti-PSMA CAR, and wherein the anti-PSMA CAR comprises an anti-PSMA scFv, a transmembrane domain, and a CD3 zeta signaling region. In one embodiment, the dose of IL2 is 100 to 720 kIU/kg/8 h. In another embodiment, the dose of IL2 is about 300 kW/kg/8 h.

In one embodiment, the IL2 is administered to the human subject by bolus infusion for four consecutive days beginning on the day of administration of the population of cells.

In one embodiment, the IL2 is administered to the human subject by bolus for five consecutive days beginning on the day of administration of the population of cells.

In one embodiment, the population of cells comprises 1×10⁸ to 1×10¹¹ cells.

In one embodiment, non-myeloablative (NMA) chemotherapy is administered to the human subject before administration of the population of cells.

In one embodiment, the population of cells comprises T-cells obtained from the subject.

In one aspect, provided herein is a method of treating prostate cancer in a subject infused with a population of cells expressing an anti-PSMA CAR, said method comprising administering IL2 to the subject according to a dosing schedule such that an IL2 plasma level of greater than 500 pg/ml is maintained in the subject for at least a week following administration of the population of cells to the subject, wherein the anti-PSMA CAR comprises an extracellular region comprising an anti-PSMA scFv, a transmembrane domain, and a CD3 zeta signaling region.

In one embodiment, the IL2 plasma level is maintained for one to two weeks following administration of the population of cells to the subject.

In one embodiment, the dosing schedule comprises administering 100 to 720 kIU/kg/8 h of IL2 to the subject by bolus infusion.

In one embodiment, the IL2 plasma level is maintained for a month following administration of the population of cells to the subject.

In one embodiment, the dosing schedule comprises administering 25,000 IU/kg/d to 300,000 IU/kg/d of IL2 to the subject. In one embodiment, the subject has an activated cell engraftment of at least 10%.

In one embodiment, the subject has an activated cell engraftment of at least 50%.

In another aspect, provided herein is a method of treating cancer in a subject who has been infused with a population of cells expressing a CAR which is specific for a cancer antigen, said method comprising administering IL2 to the subject according to a dosing schedule such that an IL2 plasma level of greater than 500 pg/ml is maintained in the subject for at least a week following administration of the population of cells to the subject, wherein the subject has received lymphodepletion therapy prior to administration of the population of cells to the subject.

In yet another aspect, provided herein is a method of treating cancer in a subject, said method comprising administering a population of cells expressing a CAR which is specific for a cancer antigen to the subject having cancer and subsequently administering IL2 to the subject either by bolus infusion comprising administering a dose of IL2 of 100 kIU/kg/8 h or more, or by continuous infusion comprising administering 25,000 IU/kg/d to 300,000 IU/kg/d of IL2 to the subject, wherein the subject has received lymphodepletion therapy prior to administration of the population of cells to the subject.

In one embodiment, the lymphodepletion therapy comprises administration of cyclophosphamide and fludarabine.

In one embodiment, the cancer is selected from the group consisting of colon cancer, breast cancer, brain cancer, lung cancer, ovarian cancer, head and neck cancer, bladder cancer, melanoma, colorectal cancer, and pancreatic cancer.

In one embodiment, the cancer antigen is selected from the group consisting of carcino-embryonic antigen (CEA), CD19, GM2, GD2, sialyl Tn (STn), HER2, EGFR, GD3, IL13R, MUC-1, and EGFRvIII.

In one embodiment, the IL2 is aldesleukin (Proleukin).

In one embodiment, the anti-PSMA scFv comprises a light chain variable region comprising the amino acid sequence as set forth in SEQ ID NO: 1, and comprising a heavy chain variable region comprising the amino acid sequence as set forth in SEQ ID NO: 2.

In one embodiment, the anti-PSMA CAR comprises a CD8 hinge region. In one embodiment, the CD8 hinge region comprises an amino acid sequence as set forth in SEQ ID NO: 4, or a functional fragment thereof.

In one embodiment, the CD3 zeta signaling region comprises an amino acid sequence as set forth in SEQ ID NO: 5, or a functional fragment thereof.

In one embodiment, the prostate cancer is associated with PSMA expression. In one embodiment, the prostate cancer is metastatic prostate cancer, recurrent prostate cancer, or hormone-refractory prostate cancer.

In one embodiment, the population of cells has been activated with an anti-CD3 antibody prior to administration to the human subject.

### FIGURES

FIGS. 1A to 1F describe the impact of conditioning. FIG. 1A. Peripheral leukocytes post-conditioning are represented as absolute neutrophil (ANC, o) and absolute lymphocyte (ALC, •) counts. Chemotherapy was from day −8 to day −2. T cells (1e9) were infused on day 0. IL2 was initiated on day 0 by continuous intravenous infusion. FIG. 1B describes dTc engraftment at day 14, time of marrow recovery. Flow cytometric profiles of dTc dose prior to patient infusion and of blood at day 14. CAR+ cells are 61% of CD3+ T cells in the dose and 7.3% of CD3+ T cells in the blood at time of marrow recovery. FIG. 1C describes a time course of dTc recovery. The fraction of dTc among CD8+ T cells in patient blood (upper) and absolute numbers of CD8+ dTc (lower) over time. Day 5 was the first day that the WBC was high enough (0.2e6/ml) to be practical to do flow. All data are from Pt 2. FIG. 1D describes a comparison of dTc pharmacokinetics with and without prior conditioning. Blood levels of total WBC and dTc in Pt 4 (solid symbols) were compared by PCR at times post-infusion with those in a patient on a second study with a different CAR (anti-CEA) (open symbols) in which conditioning was not applied. Both patients received similar-sized doses of 1-2e10 T cells with 40-50% CAR modification. Total white cells are indicated by square symbols and CAR+ T cells by round symbols. FIG. 1E. and FIG. 1F. IL15 and IL7 levels as a consequence of lymphopenic conditioning. Cytokine levels (bars) were measured in serum as in Methods at sampling time points indicated. Baseline is taken prior to chemotherapy. ALC values (solid circles, •) are plotted for comparison.

FIGS. 2A to 2C provide results relating to the combined treatment with Interleukin 2. FIG. 2A. IL2 in plasma differed markedly among patients. Serum samples were analyzed by ELISA at times after T cell infusion, expressed in pg/ml. Also represented are concentrations in IU/ml, as noted in Methods. Pt 1 had IL2 suspended after day 3 during a period of sepsis that was later resumed at half-rate on day 5 and then at full-rate on day 6 until day 28. IL2 in the infusion bag in Pts 3-5 created small serum peaks post-infusion that rapidly dissipated. FIG. 2B. Decreased plasma IL2 levels accompany higher engraftments of activated T cells. Data from Table 2B. (B1). Patient specific IL2 levels and engraftments. Going from Pt 1-5, left to right, as engrafted aTc (blue) increase, IL2 levels (red) decrease; when aTc engraftment decreases, IL2 increases. FIG. 2C. Plot of plasma IL2 as a function of aTc engrafted. Inset: log regression: more aTc, less IL2, with correlation coefficient=−0.94 and p<0.01.

FIGS. 3A-3C. PSA response. FIGS. 3A and 3B. PSA after dTc infusion in two partial responders (FIG. 3A: Pt 1; FIG. 3B: Pt 2). Chemotherapy conditioning took place between day −8 and day −2. Day 0 (arrow) was time of dTc infusion. FIG. 3C. PSA delays after dTc infusion. PSA data preceding dTc dosing were analyzed for all patients by semi-log plot to determine the PSA trajectory prior to treatment (solid line). This was a period that was uninterrupted by any new therapies. (Patients progressing on ADT were continued on ADT.) Arrow marked “Chemo” is blood sample for PSA drawn on admission to hospital for cyclophosphamide, before chemotherapy administration. Arrow marked “T cells” is blood sample for PSA drawn on admission to hospital for dTc infusion, but prior to infusion. Post dTc infusion, only those values obtained before other intervention are represented; arrows indicate onset of new therapy (“Ketoconazole”). The PSA delay is estimated as the time interval from the value projected on the solid line that equals the final PSA value before a new treatment. A PSA delay was evident only in patients 1, 2 and 5.

FIGS. 4A and 4B describe data showing a lack of anti-CAR response in patient sera. FIG. 4A provides data showing control staining for CAR+ controls. Anti-CEA CAR+ Jurkat cells reacted with human CEA-Fc, detected with goat anti-human Ig secondary antibody (Ab) to show secondary Ab detects human Fc reacting with CAR+ cells. Anti-PSMA CAR+ Jurkat cells reacted with anti-V5 Ab (mouse), detected with goat anti-mouse Ig secondary Ab to show the profile to expect if there are positive sera among patients treated in the study described in the Example. FIG. 4B provides data showing results from patients' post-treatment serum sample screening for anti-CAR antibody. Patient 1-5 (P1 to P5) sera were collected at times 1 to 6 months post dTc infusion and incubated with anti-PSMA CAR+ Jurkat cells and examined by flow cytometry. No anti-CAR reactivity was detected. Jurkat PSMA CAR was stained with serum then anti-human Ig PE.

FIG. 5 shows provides data showing proliferation of dTc on PSMA+ targets. Unmodified (T) or IgTCR-modified T cells were mixed 1:1 with irradiated tumor cells on day 0. T cell counts were recorded at times indicated. The left panel shows PC3 and the right panel shows PC3-PSMA. Co-cultures of dTc and PC-PSMA led to lysis and clearing of all targets, but had no effect on antigen-negative PC3 targets.

FIG. 6 describes qPCR results for anti-PSMA dTc (left) and Albumin (right). The upper panels describe the fluorescence profiles versus cycles for standards and unknowns. The middle panels describe the melt-curves showing high quality PCR products. The lower panels describe the determination values of unknowns versus standard curves.

FIG. 7 provides a schematic of the anti-PSMA CAR used in the Example.

### DETAILED DESCRIPTION OF THE INVENTION

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art.

As used herein, the term “Chimeric Antigen Receptor” or “CAR” refers to a recombinant fusion protein comprising at least an extracellular antigen-binding protein, a trans membrane domain, and an intracellular signaling domain (also referred to as a cytoplasmic signaling domain) derived from a stimulatory molecule as defined below. In one embodiment, the extracellular antigen-binding domain is composed of a single chain variable fragment (scFv or sFv) comprising a variable heavy region and a variable light region of an antibody.

The term “signaling domain” or “signaling region”, as used interchangeably herein refer to the functional portion of a protein which acts by transmitting information within the cell to regulate cellular activity via defined signaling pathways by generating second messengers or functioning as effectors by responding to such messengers.

As used herein, the term “PSMA” refers to Prostate Specific Membrane Antigen, which is an antigenic determinant detectable on prostate tissue, including carcinoma. The human amino acid and nucleic acid sequences can be found in a public database, such as GenBank, UniProt and Swiss-Prot. For example, the amino acid sequence of human PSMA can be found as UniProt/Swiss-Prot Accession No. Q04609.1 and the NCBI Reference Sequence ID number for the amino acid sequence of human PSMA is NP_004467.1. The nucleotide sequence encoding human PSMA can be found at Accession No. NM_004476.1. The amino acid sequence of the extracellular region of human PSMA is provided below as SEQ ID NO: 6.

(SEQ ID NO: 6)
SSNEATNITPKHNMKAFLDELKAENIKKFLYNFTQIPHLAGTEQNFQLAK
QIQSQWKEFGLDSVELAHYDVLLSYPNKTHPNYISIINEDGNEIFNTSLF
EPPPPGYENVSDIVPPFSAFSPQGMPEGDLVYVNYARTEDFFKLERDMKI
NCSGKIVIARYGKVFRGNKVKNAQLAGAKGVILYSDPADYFAPGVKSYPD
GWNLPGGGVQRGNILNLNGAGDPLTPGYPANEYAYRRGIAEAVGLPSIPV
HPIGYYDAQKLLEKMGGSAPPDSSWRGSLKVPYNVGPGFTGNFSTQKVKM
HIHSTNEVTRIYNVIGTLRGAVEPDRYVILGGHRDSWVFGGIDPQSGAAV
VHEIVRSFGTLKKEGWRPRRTILFASWDAEEFGLLGSTEWAEENSRLLQE
RGVAYINADSSIEGNYTLRVDCTPLMYSLVHNLTKELKSPDEGFEGKSLY
ESWTKKSPSPEFSGMPRISKLGSGNDFEVFFQRLGIASGRARYTKNWETN
KFSGYPLYHSVYETYELVEKFYDPMFKYHLTVAQVRGGMVFELANSIVLP
FDCRDYAVVLRKYADKIYSISMKHPQEMKTYSVSFDSLFSAVKNFTEIAS
KFSERLQDFDKSNPIVLRMMNDQLMFLERAFIDPLGLPDRPFYRHVIYAP
SSHNKYAGESFPGIYDALFDIESKVDPSKAWGEVKRQIYVAAFTVQAAAE
TLSEVA

In one aspect the antigen-binding portion of the CAR recognizes and binds an epitope within the extracellular domain of the PSMA protein, or fragments thereof. As used herein, “PSMA” includes proteins comprising mutations, e.g., point mutations, fragments, insertions, deletions and splice variants of full length wild-type PSMA.

As used herein, the term “antigen binding protein” refers to a protein or polypeptide that can specifically bind to a target molecule, such as prostate specific membrane antigen (PSMA). An antibody is an example of an antigen binding protein. An scFv is another example of an antigen binding protein. Preferably, the extracellular region of a CAR comprises an antigen binding protein.

The term “cancer antigen” as used herein can be any type of cancer antigen known in the art. A preferred cancer antigen is a cell surface antigen, such as, but not limited to, PSMA. In some embodiments, the term cancer antigen refers to an antigen that is aberrantly expressed in, mutated in, or specific to, a cancer cell.

An “epitope” is the portion of a molecule that is bound by an antigen binding protein (e.g., by an antibody or scFv). In one embodiment, an epitope comprises non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding protein). Generally the variable regions, particularly the CDRs, of an antigen binding protein interact with the epitope.

The term “antibody” refers to an immunoglobulin (Ig) molecule comprised of four polypeptide chains, two heavy (H) chains and two light (L) chains, or any functional fragment, mutant, variant, or derivation thereof, which retains the essential epitope binding features of an Ig molecule.

Generally, the amino-terminal portion of each antibody chain includes a variable region that is primarily responsible for antigen recognition. The carboxy-terminal portion of each heavy and light chain of an antibody comprises a constant region, e.g., responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)). The variable regions of each light/heavy chain pair form the antibody binding site such that an intact immunoglobulin has two binding sites. A single VH or VL domain may be sufficient to confer antigen-binding specificity.

The variable regions of antibody heavy and light chains (VH and VL, respectively) exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is known in the art, including, for example, definitions as described in Kabat et al. in Sequences of Proteins of Immunological Interest, 5^{th} Ed., US Dept. of Health and Human Services, PHS, NIH, NIH Publication no. 91-3242, 1991 (herein referred to as “Kabat numbering”). For example, the CDR regions of an antibody can be determined according to Kabat numbering.

An “antibody fragment”, “antibody portion”, “antigen-binding fragment of an antibody”, or “antigen-binding portion of an antibody” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies.

A Fab fragment is a monovalent antibody fragment having the VL, VH, CL and CH1 domains; a F(ab′)₂ fragment is a bivalent fragment having two Fab fragments linked by a disulfide bridge at the hinge region; a Fd fragment has the VH and CH1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634; 6,696,245, US App Pub 20/0202512; 2004/0202995; 2004/0038291; 2004/0009507; 2003/0039958, and Ward et al., Nature 341:544-546, 1989).

In one embodiment, the antigen binding protein is a single-chain antibody (scFv or sFv). An scFv refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker. An scFv is capable of being expressed as a single chain polypeptide, wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “specifically binds,” as used herein with respect to an antigen binding protein, refers to the ability of an antigen binding protein, e.g., an scFv, to form a complex with an antigen that is relatively stable under physiologic conditions.

The terms “anti-PSMA antibody” or “anti-PSMA scFv” refer to an antibody or scFv, respectively, that specifically binds PSMA. Similarly, the term “anti-PSMA CAW” refers to a CAR that specifically binds to PSMA. Preferably, the PSMA is human PSMA.

As used herein, the term “nucleic acid” or “polynucleotide”, used interchangeably herein, refers to deoxyribonucleic acids (DNA) or ribonucleic acids (RNA), and polymers thereof, in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; and Rossolini et al. (1994) Mol. Cell. Probes 8:91-98).

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters.

Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “host cell” refers to any cell that has been modified, transfected, transformed, and/or manipulated in any way to express an anti-PSMA-CAR as disclosed herein. For example, in some embodiments, the host cell has been modified to comprise an exogenous polynucleotide (e.g., a vector, linear DNA molecule, mRNA) encoding an anti-PSMA-CAR disclosed herein. In one embodiment, the host cell is a human cell. In some embodiments, the hostcell is an immune cell. In some embodiments, the immune cell is selected from the group consisting of a dendritic cell, a mast cell, an eosinophil, a T cell (e.g., a regulatory T cell), a B cell, a cytotoxic T lymphocyte, a macrophage, a monocyte, and a Natural Killer (NK) T cell. In some embodiments the host cell is a T cell, e.g., a T cell obtained from a subject having cancer, e.g, prostate cancer. In one embodiment, a host cell is an autologous T cell.

The term “transfected” or “transformed” or “transduced” as used herein refers to a process by which exogenous nucleic acid is transferred or introduced into a host cell. A “transfected” or “transformed” or “transduced” cell is one which has been transfected, transformed or transduced with exogenous nucleic acid. The cell includes the primary subject cell and its progeny.

As used herein, the term “high expression level” refers to a level of a molecular marker (e.g., a protein and/or an RNA (e.g., a mRNA)) which is increased in a disease state in a subject (or sample thereof) relative to a normal level, i.e., that of a healthy subject who does not have the disease. In one embodiment, the high level of expression refers to a level which is associated with cancer in a subject, e.g., a high expression level of a cancer antigen.

The term “recombinant protein” refers to a protein that is expressed from a cell or cell line transfected with an expression vector (or possibly more than one expression vector) comprising the coding sequence of the protein (e.g., a DNA sequence encoding the protein). In one embodiment, said coding sequence is not naturally associated with the cell. For example, a human protein, such as human IL2, could be produced in bacteria, e.g., E. coli, and, therefore, have a different glycosylation pattern than IL2 as it is found in humans. In one embodiment, a recombinant protein is recombinant human IL2.

As used herein, the term “subject” includes human and non-human animals. Non-human animals include all vertebrates (e.g., mammals and non-mammals) such as, mice, rats, rabbits, humans, non-human primates, sheep, horses, dogs, cats, cows, chickens, amphibians, and reptiles. Except when noted, the terms “patient” or “subject” are used herein interchangeably. In a preferred embodiment, the subject is a human male subject.

As used herein, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 20%, 15%, 10%, 9%, 8%, 7%, 6%, 5%, 4%, 3%, 2%, 1%, 0.5%, 0.1%, or 0.05% of a given value or range.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated.

To “treat” a disease as the term is used herein, means to reduce the frequency or severity of at least one sign or symptom of a disease or disorder experienced by a subject.

As used herein, the term “cancer” refers to or describes the physiological condition in mammals that is typically characterized by unregulated cell growth. An example of a type of cancer is prostate cancer.

It should be noted that where amino acid sequences are described throughout, it is also contemplated that nucleic acids encoding said proteins are included in the invention. Further, where it is indicated that a host cell expresses a CAR having a specific amino acid sequence, it is also contemplated herein that the host cell is transduced with a nucleic acid encoding the CAR.

### Methods of Invention

The invention provides a combination therapy based on the use of interleukin-2 (IL2) and designer T cells (also referred to herein as chimeric antigen receptor (CAR) T cells) to treat a human subject having cancer, such as prostate cancer. The invention is based, at least in part, on the surprising discovery that there is a correlation between IL2 plasma levels in a subject and levels of activated T cell engraftment following administration of a population of T cells expressing a CAR directed against a cancer antigen, such as PSMA. It should be noted that where a population of cells expressing a cancer-specific CAR is described, it is intended to refer to a population of cells wherein individual cells express the CAR.

Included in the invention is a method of treating cancer in a subject who has been infused with a population of cells expressing a CAR which is specific for a cancer antigen. The subject is administered IL2 according to a dosing schedule such that an IL2 plasma level of greater than 500 pg/ml is maintained in the subject for at least a week following administration of the population of cells to the subject. In one embodiment, prior to the administration of the population of CAR-expressing cells opt the subject, the subject receives lymphodepletion therapy.

In one embodiment, the invention features a method of treating cancer comprising administering a population of cells expressing a CAR which is specific for a cancer antigen to the subject having cancer and subsequently administering IL2 to the subject either by bolus infusion comprising administering a dose of IL2 of 100 kIU/kg/8 h or more, or by continuous infusion comprising administering 25000 IU/kg/d to 300000 IU/kg/d of IL2 to the subject.

In one embodiment, the subject also received lymphodepletion therapy, e.g., NMA conditioning, in combination with the CAR cell transduction and IL2 therapy. As described in the example below, such conditioning provides therapeutic advantages with the CAR/IL2 combination therapy. Thus, a subject having cancer may receive lymphodepletion therapy comprising administration of cyclophosphamide and fludarabine. Such therapy is usually performed in the days prior to administration of the population of CAR expressing cells to the subject.

The methods disclosed herein may be used to treat any cancer which can be targeted by a CAR, i.e., a cell surface antigen. Examples of cancer that may be treated using the methods disclosed herein include, but are not limited to, colon cancer, prostate cancer, breast cancer, brain cancer, lung cancer, ovarian cancer, head and neck cancer, bladder cancer, melanoma, colorectal cancer, and pancreatic cancer. Further, examples of cancer antigens that CARs used in the invention may bind to include, but are not limited to, carcino-embryonic antigen (CEA), CD19, GM2, GD2, sialyl Tn (STn), HER2, EGFR, GD3, IL13R, MUC-1, PSMA, and EGFRvIII.

While the example below and description herein refer to anti-PSMA CARs and prostate cancer, this CAR and cancer type are not intended to be limiting. As described above, the methods and compositions described herein are useful for many types of cancer that are associated with a cell surface antigen, as well as a CAR that can bind said cancer antigen.

The treatment method described herein provides, at least in part, sustained IL2 levels in an engraftment setting in a subject having prostate cancer. Continuous infusion of IL2 or a bolus administration of IL2 is used to sustain the activation state of PSMA-CAR transduced cells in high engraftment settings while preserving patient tolerance of the regimen. As described in the Example below, the data show that certain doses of IL2 are beneficial for maintaining activation of anti-PSMA CAR-T cells, resulting in a positive clinical response. Thus, the invention provides a combination method for treating prostate cancer comprising administering a population of cells transduced with a nucleic acid encoding an anti-PSMA CAR to a subject and administering IL2 to the subject, wherein the amount of IL2 is sufficient to maintain activation of anti-PSMA CAR T cells infused into the patient.

IL2 is a secreted cytokine which is involved in immunoregulation and the proliferation of T and B lymphocytes. IL2 has been shown to have a cytotoxic effect on tumour cells and recombinant human IL2 (aldesleukin/Proleukin™) has FDA approval for treatment of metastatic renal carcinoma and metastatic melanoma. IL2 as a therapeutic agent has little impact on prostate cancers; its primary utility has been demonstrated in renal cell carcinoma and melanoma. The experiments described herein describe a correlation between the level of plasma IL2 and clinical response in patients who received anti-PSMA CAR treatment for prostate cancer. Accordingly, IL2, e.g., aldesleukin (Proleukin), is used in the methods of the invention to support the survival and expansion of gene-modified T cells specific for PSMA. In one embodiment, the methods described herein use an IL2 protein as set forth in the amino acid sequence of SEQ ID NO: 8, provided below.

### Amino Acid Sequence of Des-Alanyls-1, Serine 125 Human IL2.

(SEQ ID NO: 8)
PTSSSTKKTQLQLEHLLLDLQMILNGINNYKNPKLTRMLTFKFYMPKKAT
ELKHLQCLEEELKPLEEVLNLAQSKNFHLRPRDLISNINVIVLELKGSET
TFMCEYADETATIVEFLNRWITFSQSIISTLT

The amino acid sequence of mature human IL2 is set out in SEQ ID NO: 7, provided below, and publicly available under the Swiss Prot database as P60568.

### Amino Acid Sequence of Human IL2

(SEQ ID NO: 7)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

The IL2 used in the invention may comprise a sequence of all or functional fragment of the IL2 amino acid sequence shown in SEQ ID NO: 7. Variants of the SEQ ID NO: 7 amino acid sequence may be used, e.g. natural variants encoded by human alleles and/or variants with one or two amino acid mutations. A mutation may be deletion, substitution, addition or insertion of an amino acid residue. In one embodiment, IL2 used herein is recombinant IL2.

IL2, or a functional fragment thereof, used in the present invention may have at least 90% sequence identity, at least 95% sequence identity or at least 98% sequence identity to the mature human IL2 sequence set out in SEQ ID NO: 7. Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST. Sequence identity may be determined with reference to the full length of a sequence set out herein.

A functional fragment or variant version (e.g., 95% identity or more) of IL2 preferably retains the activity of full length human IL2. For example, in one embodiment a functional fragment or variant of IL2 used herein his able to induce killer cell activity (e.g., lymphokine-activated (LAK) and natural (NK) activity) or is able to induce interferon gamma production.

In one embodiment of the invention, a continuous infusion of IL2 is administered to a human subject having prostate cancer following administration of anti-PSMA CAR expressing cells. For example, IL2 may be administered to the human subject by continuous intravenous infusion at a dose of 25000 to 300000 IU/kg/d. In one embodiment, IL2 is administered to the human subject by continuous intravenous infusion at a dose of 50000 to 200000 IU/kg/d. In one embodiment, IL2 is administered to the human subject by continuous intravenous infusion at a dose of 50000 to 200000 IU/kg/d. In one embodiment, IL2 is administered to the human subject by continuous intravenous infusion at a dose of 75000 to 100000 IU/kg/d. In one embodiment, IL2 is administered to the human subject by continuous intravenous infusion at a dose of about 75000 IU/kg/d. The IL2 may be administered to the subject by continuous intravenous infusion. In one embodiment, IL2 is administered continuously as an infusion for about 20-30 days; 21-31 days; 21-29 days; or 22-28 days. In one embodiment, IL2 is administered as a continuous infusion for 7 days, 28 days, a month, two months, or three months. Dose levels of IL2 by continuous infusion have been estimated to maintain blood levels in the range of 25-40 IU/ml, which assures >98% saturation of the high affinity IL2R on the activated CAR T cells. The methods described herein are useful for maintaining IL2 at a tolerable level for one month following the T cell dose, such that more sustained anti-tumor T cell response can be achieved resulting in, for example, a clinical response, e.g., a decrease in prostate specific antigen (PSA) levels.

Alternatively, IL2 may be administered intravenously to a human subject having prostate cancer at a dose of 100 kIU/kg/8 h or more, where the IL2 is administered after administration of a population of cells expressing an anti-PSMA CAR. In one embodiment, the dose of IL2 is 100 to 720 kIU/kg/8 h or about 300 kIU/kg/8 h. When administered at this higher dose, IL2 may be administered intravenously as a bolus for four consecutive days or longer as tolerated. A bolus of IL2 may also be administered at a dose of 100 kIU/kg/8 h or more (e.g., 100 to 720 kIU/kg/8 h) for five consecutive days, six consecutive days, seven consecutive days and so forth. In one embodiment, the dose of IL2 is 200 to 720 kIU/kg/8 h; 200 to 500 kIU/kg/8 h; 250 to 400 kIU/kg/8 h; 300 to 500 kIU/kg/8 h; or 300 to 400 kW/kg/8 h.

In one embodiment, administration of IL2 to the subject is initiated on the same day as administration of the population of cells expressing a PSMA-CAR. In an alternative embodiment, IL2 administration is initiated one day, two days, three days, four days, five days, or six days after infusion of the PSMA-CAR expressing cells to the subject.

The dose of IL2 that is administered in a combination therapy with PSMA-CAR expressing cells (e.g., T cells) is, in some embodiments, an amount of IL2 that is effective for achieving a peak plasma concentration of at least 2000 pg/ml within the first week following initiation of the IL2 treatment. In an alternative embodiment, a human subject is administered an amount of IL2 that is effective for maintaining a plasma level of 500 pg/ml, 750 pg/ml, or 1000 pg/ml or more during treatment with IL2.

Indeed, the invention is based, at least in part, on the discovery that PSMA-CAR expressing T cells maintain anti-tumor activity and activation in a human subject in the presence of a certain plasma level of IL2. As described in the Example below, a plasma level of IL2 of a subject (who was administered T cells expressing a PSMA-CAR) below about 500 pg/ml results in decreased anti-tumor activity. Such activity can be determined, for example, by measuring a marker associated with prostate cancer, such as prostate specific antigen (PSA). PSA is also a marker for determining clinical response.

The methods described herein are beneficial for achieving an activated cell engraftment of at least 10%, of at least 20%, of at least 30%, or, in certain embodiments, an activated cell engraftment of at least 50%. As was observed in the Example below, there is a direct correlation between plasma levels of IL2 in a subject and the clinical response for prostate cancer treatment, where peak plasma levels 1500 pg/ml or greater correlated with a positive clinical response. Thus, the plasma level of IL2 in a subject who has received an infusion of PSMA-CAR expressing cells can be assessed, for example, within a day or within a week of initiating IL2 therapy following the CAR T cell infusion. If the peak level is determined to be low, e.g., less than 500 pg/ml, then additional IL2 should be administered to the subject.

IL-2 may be administered to the subject using methods known in the art. For example, IL-2 may be administered to a subject transarterially, subcutaneously, intradermally, intratumorally, intranodally, intramedullary, intramuscularly, by intravenous (i.v.) injection, or intraperitoneally. In one embodiment, IL-2 is administered to a subject by subcutaneous injection. In another embodiment, IL-2 is administered to a subject intravenously. IL-2 may also be administered to a subject via continuous infusion or by bolus infusion.

In one embodiment, the invention features a method of treating prostate cancer in a subject who has been infused with a population of cells expressing an anti-PSMA CAR, where the method comprises administering IL2 to the subject according to a dosing schedule such that an IL2 plasma level of greater than 500 pg/ml is maintained in the subject for at least a week following administration of the population of cells to the subject. In one embodiment, the IL2 plasma level of the subject is maintained for one to two weeks following administration of the population of cells to the subject. In another′ embodiment, the dosing schedule comprises administering 100 to 720 kIU/kg/8 h of IL2 to the subject in order to maintain a desired IL2 plasma level which has been discovered as being advantageous for maintaining activated T cells expressing PSMA-CARs. In a further embodiment, the dosing schedule comprises administering about 75000 IU/kg/d of IL2 to the subject.

In one aspect, the present invention provides a method for inhibiting the proliferation or reducing the population of cancer cells expressing PSMA in a subject, the method comprising contacting the cancer-associated antigen-expressing cell or cell population with a host cell comprising an anti-PSMA CAR followed by administration of IL2 to the subject, thereby inhibiting the proliferation or reducing the population of cancer cells expressing PSMA. In certain aspects, the method results in a reduction in the quantity, number, amount or percentage of malignant and/or cancer cells by at least 25%, at least 30%, at least 40%, at least 50%, at least 65%, at least 75%, at least 85%, at least 95%, or at least 99% in a subject, as compared to the quantity, number, amount or percentage of malignant and/or cancer cells in a subject prior to administering the host cell.

The methods of the invention include administration of a population of host cells expressing an anti-PSMA CAR in order to treat prostate cancer. A population of cells (or a composition comprising said population) includes a number of cells that is effective at providing treatment for prostate cancer when used in the combination methods of the invention. In one embodiment, the population of cells comprises about 1×10⁸ to about 5×10¹¹ cells; alternatively, the population comprises about 5×10⁸ to about 5×10¹¹ cells; about 1×10⁹ to about 1×10¹¹ cells; about 5×10⁹ to about 1×10¹¹ cells; about 5×10⁹ to about 5×10¹⁰ cells; or about 5×10⁹ to about 5×10¹¹ cells. In some embodiments, about 10², 10³, 10⁴, 10⁵, 10⁶, 10⁷, 10⁸, 10⁹, 10¹⁰, 10¹¹, 10¹², or more, host cells comprising a nucleic acid encoding an anti-PSMA CAR described herein are administered to a subject. Host cell compositions may also be administered multiple times at these dosages.

A population of transduced host cells may be administered to a subject by any means known in the art, including transfusion, implantation or transplantation. In a preferred embodiment, a population of host cells expressing an anti-PSMA CAR is administered to a subject by infusion, e.g., bolus or slow infusion.

In one embodiment, the population of cells has been activated with an anti-CD3 antibody prior to administration to the human subject. In another embodiment, the cells are activated with anti-CD3 anti-CD28 beads.

In one embodiment, the population of cells has been conditioned with IL-12 prior to administration to the human subject (see, e.g., Emtage et al. (2003) J. Immunother. 16(2): 97-106, incorporated herein by reference).

Combination methods disclosed herein include administration of therapeutic agents in combination with a composition comprising transduced host cells comprising an expression vector encoding an anti-PSMA CAR, wherein the therapeutic agent is administered before, after or concurrently with the composition of transduced cells. An example of a therapeutic agent is IL2. An alternative additional therapeutic agent is a chemotherapeutic agent.

In one embodiment, non-myeloablative (NMA) chemotherapy is administered to the human subject before administration of the population of cells. NMA conditioning is used to induce stable engraftment of the infused autologous anti-PSMA CAR cells. This engraftment then affords the opportunity of supporting a sustained anti-tumor response. Thus, infusion of the cells after NMA conditioning provides advantageous for improved treatment of the cancer. Such NMA methods are known in the art, including Dudley et al. (2002) Science. 298:850-4. Thus, in one embodiment, a human subject undergoes NMA conditioning prior to infusion of the anti-PSMA-CAR cells. NMA conditioning includes administration of cyclophosphamide and fludarabine prior to infusion of the cells. In a preferred embodiment, cyclophosphamide and fludarabine are each administered to the human subject within 10 days prior to infusion of the anti-PSMA-CAR cells to the subject. For example, cyclophosphamide can be administered for two days, e.g., at days −8 and −7 prior to infusion (the infusion day being zero) and fludarabine can be administered to the subject for five consecutive days from day −6 to day −2. In one embodiment, 60 mg/kg of cyclophosphamide is administered to the subject. In one embodiment, 25 mg/m² of fludarabine is administered to the subject. In one embodiment, there is a day of no treatment on day −1, the day immediately prior to the anti-PSMA CAR cell infusion to the subject. In one embodiment, the subject is administered a combination therapy of cyclophosphamide and fludarabine (as separate agents) wherein cyclophosphamide and fludarabine are administered to the subject on individual days (i.e., are administered to the subject on a day when the other agent is not administered), prior to the day of infusion of the transduced cells which is also the day that IL2 therapy is initiated.

In some aspects of the invention, the host cells expressing anti-PSMA CARs are administered to a subject, such that the host cells (or their progeny), persist in the subject for a given number of days, including, but not limited to, at least 0.5 days, one day, two days, three days, four days, five days, six days, seven days, eight days, nine days, ten days, eleven days, twelve days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, thirty-one days or more, after administration of the host cell to the subject.

The methods disclosed herein are useful for treating prostate cancer. In one embodiment, the prostate cancer is associated with high levels of expression of PSMA. Examples of types of prostate cancer that can be treated using the methods disclosed herein include, but are not limited to, metastatic prostate cancer, recurrent prostate cancer, or hormone-refractory prostate cancer.

Chimeric Antigen Receptor (CAR) that Binds Cancer Antigen

The methods disclosed herein are based, at least in part, on the administration of host cells expressing chimeric antigen receptors (CARs) that are specific for a cancer antigen. In one embodiment, the methods disclosed herein are based, at least in part, on the administration of host cells expressing PSMA-specific chimeric antigen receptors (CARs).

CARs are synthetic, engineered receptors that can target surface molecules in their native conformation. Unlike TCRs, CARs engage molecular structures independent of antigen processing by the target cell and independent of MHC. CARs typically engage the target via a single-chain variable fragment (scFv) derived from an antibody.

A CAR generally contains an extracellular region, e.g., a single chain variable fragment (scFv) of an antibody recognizing a tumor antigen (such as PSMA), a transmembrane domain, and an intracellular region, e.g., a T-cell receptor (TCR) zeta chain that mimics TCR activation. A CAR may also further comprise an intracellular signaling domain derived from CD28 or 4-IBB to mimic co-stimulation. Thus, CARs are generally constructed by joining the antigen recognition domains of an antibody with the signaling domains of receptors from T cells. Modification of T cells with nucleic acid sequences encoding CARs equips T cells with retargeted antibody-type antitumor cytotoxicity. Because killing is MHC-unrestricted, the approach offers a general therapy for all patients bearing the same antigen. These T cells engineered with artificial CARs are often called “designer T cells”, “CAR-T cells,” or “T-bodies” (Eshhar et al. Proc. Natl. Acad. Sci. USA 90(2): 720-724, 1993; Ma et al. Cancer Chemother. Biol. Response Modif. 20: 315-41, 2002).

In one embodiment, anti-PSMA CARs as described in US 2007/0031438, which is incorporated by reference herein, are used in the methods of the invention.

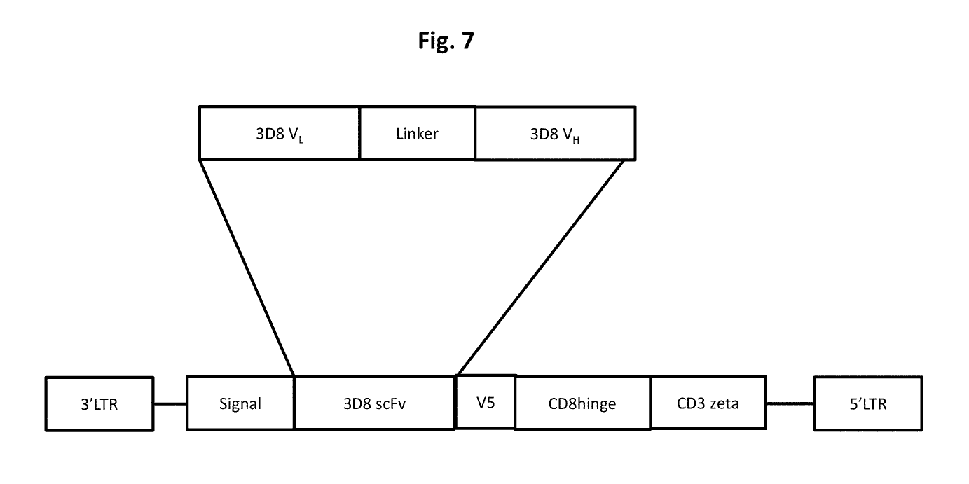

An exemplary CAR for use in the invention is also provided in FIG. 7.

### Extracellular Antigen Binding Region of CAR

The present invention pertains, in part, to methods of treatment using CARs that bind to a cancer antigen, such as PSMA, e.g., human PSMA. Thus, in one aspect, the antigen binding region of aCAR comprises an antigen binding protein that binds to a cancer antigen. For example, the extracellular region of a CAR used in the methods of the invention may comprise an antigen binding protein, such as an scFv, that binds a cancer antigen selected from one of the following: Further, carcino-embryonic antigen (CEA), CD19, GM2, GD2, sialyl Tn (STn), HER2, EGFR, GD3, IL13R, MUC-1, PSMA, and EGFRvIII. In one embodiment, the antigen binding region of the anti-PSMA CAR comprises an antigen binding protein that binds to PSMA.

In one embodiment, the invention provides an anti-PSMA CAR comprising an extracellular region comprising an antigen binding protein that binds to PSMA, wherein the antigen binding protein comprises a heavy chain variable (VH) domain comprising an amino acid sequence that is at least 95% identical to the amino acid sequence of SEQ ID NO: 2. In one embodiment, the anti-PSMA CAR comprises an extracellular region comprising a VH domain comprising an amino acid sequence that is at least 96% identical to the amino acid sequence of SEQ ID NO: 2. In one embodiment, the anti-PSMA CAR comprises an extracellular region comprising a VH domain comprising an amino acid sequence that is at least 97% identical to the amino acid sequence of SEQ ID NO: 2. In one embodiment, the anti-PSMA CAR comprises an extracellular region comprising a VH domain comprising an amino acid sequence that is at least 98% identical to the amino acid sequence of SEQ ID NO: 2. In one embodiment, the anti-PSMA CAR comprises an extracellular region comprising a VH domain comprising an amino acid sequence that is at least 99% identical to the amino acid sequence of SEQ ID NO: 2. In one embodiment, the anti-PSMA CAR comprises an extracellular region comprising a VH domain comprising the amino acid sequence of SEQ ID NO: 2. In a further embodiment, the anti-PSMA CAR comprises an extracellular region comprising the CDRs set forth in SEQ ID NO: 2 (according to Kabat numbering).

In one embodiment, the invention provides an anti-PSMA CAR comprising an extracellular region comprising an antigen binding protein that binds to PSMA, wherein the antigen binding protein comprises a light chain variable (VL) domain comprising an amino acid sequence that is at least 95% identical to the amino acid sequence of SEQ ID NO: 1. In one embodiment, the anti-PSMA CAR comprises an extracellular region comprising a VL domain comprising an amino acid sequence that is at least 96% identical to the amino acid sequence of SEQ ID NO: 1. In one embodiment, the extracellular region of the anti-PSMA CAR comprises a VL domain comprising an amino acid sequence that is at least 97% identical to the amino acid sequence of SEQ ID NO: 1. In one embodiment, the extracellular region of the anti-PSMA CAR comprises a VL domain comprising an amino acid sequence that is at least 98% identical to the amino acid sequence of SEQ ID NO: 1. In one embodiment, the extracellular region of the anti-PSMA CAR comprises a VL domain comprising an amino acid sequence that is at least 99% identical to the amino acid sequence of SEQ ID NO: 1. In one embodiment, the extracellular region of the anti-PSMA CAR comprises a VL domain comprising the amino acid sequence of SEQ ID NO: 1.

In one embodiment, the extracellular portion of a CAR used herein comprises an extracellular domain comprising antigen binding regions from the antibody 3D8.

In one embodiment, the anti-PSMA CAR comprises an anti-PSMA scFv, or a functional portion thereof; a CD8 hinge region, or a functional portion thereof; and a CD3 zeta signaling region, or a functional portion thereof; wherein the anti-PS MA scFv comprises a light chain variable region comprising the amino acid sequence as set forth in SEQ ID NO: 1, and a heavy chain variable region comprising the amino acid sequence as set forth in SEQ ID NO: 2; wherein the CD8 hinge region, or a functional portion thereof, comprises the amino acid sequence as set forth in SEQ ID NO: 4; and wherein the CD3 zeta signaling region, or a functional portion thereof, comprises any one of the amino acid sequences set forth in SEQ ID NOs: 5, 11, 12, 13, and 14. Optionally, the anti-PSMA CAR may include a V5 tag, for example, a V5 tag comprising the amino acid sequence set forth in either SEQ ID NO: 3 or SEQ ID NO: 9. Optionally, the anti-PSMA CAR may include an N-terminal signal peptide, for example, the signal peptide set forth in SEQ ID NO: 10.

In one embodiment, the anti-PSMA CAR comprises an anti-PSMA scFv, or a functional portion thereof; a CD8 hinge region, or a functional portion thereof; and a CD28 signaling region, or a functional portion thereof; wherein the anti-PSMA scFv comprises a light chain variable region comprising the amino acid sequence as set forth in SEQ ID NO: 1, and a heavy chain variable region comprising the amino acid sequence as set forth in SEQ ID NO: 2; wherein the CD8 hinge region, or a functional portion thereof, comprises the amino acid sequence as set forth in SEQ ID NO: 4; and wherein the CD28 signaling region, or a functional portion thereof, comprises any one of the amino acid sequences set forth in SEQ ID NOs: 15, 16, 17, 18, and 19.

Optionally, the anti-PSMA CAR may include a V5 tag, for example, a V5 tag comprising the amino acid sequence set forth in either SEQ ID NO: 3 or SEQ ID NO: 9. Optionally, the anti-PSMA CAR may include an N-terminal signal peptide, for example, the signal peptide set forth in SEQ ID NO: 10.

In one embodiment, the substitutions made within a heavy or light chain that is at least 95% identical (or at least 96% identical, or at least 97% identical, or at least 98% identical, or at least 99% identical) are conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kura et al., 2001, Biomol. Eng. 18:95-108).

In one embodiment, the scFv comprises a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally-occurring amino acid. In one embodiment, the linker sequence comprises amino acids glycine and serine. In one embodiment, the linker sequence comprises glycine and serine repeats, such as (Gly₄Ser)ₙ, where n is a positive integer equal to or greater than 1 (SEQ ID NO: 31). In one embodiment, the linker is (Gly₄Ser)₄ (SEQ ID NO: 23) or (Gly₄Ser)₃ (SEQ ID NO: 22). Variation in the linker length may retain or enhance activity, giving rise to superior efficacy in activity studies. In one embodiment, the linker sequence is the amino acid sequence GGSGSGGSGSGGSGS (SEQ ID NO: 21).

By combining different VL and VH-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol. Biol. 178:379-87.

In one embodiment, the invention provides an anti-PSMA CAR that comprises an extracellular region which is an anti-PSMA scFv comprising a light chain having a variable domain comprising an amino acid sequence as set forth in SEQ ID NO: 1; and a heavy chain having a variable domain comprising an amino acid sequence as set forth in SEQ ID NO: 2. The amino acid sequences of SEQ ID NOs: 1 and 2 are provided below.

### Amino Acid Sequence of Light Chain Variable Region of Antibody 3D8

(SEQ ID NO: 1)
MSPAQFLFLLVLWIQETNGDVVMTQTPLTLSVTIGQPASISCKSSQSLLY
SNGKTYLNWLLQRPGQSPKRLIYLVSKLDSGVPDRFTGSGSGTDFTLKIS
RVEAEDLGVYYCVQGTHFPHTFGGGTKLEIKR

### Amino Acid Sequence of Heavy Chain Variable Region of Antibody 3D8

(SEQ ID NO: 2)
MNFGLSLIFLVLVLKGVQCEVKVVESGGGLVKPGASLKLSCAASGFTFSN
YGMSWVRQTSDKRLEWVASISSGGDSTFYADNVKGRFTISRENAKNTLYL
QMSSLKSEDTALYYCARDDLFNWGQGTTLTVSS

In one embodiment, the invention provides an anti-PSMA CAR that comprises an antigen binding protein, such as an scFv, comprising a light chain having a complementarity determining region (CDR) set (meaning a CDR1, a CDR2, and a CDR3) corresponding to a variable domain comprising an amino acid sequence as set forth in SEQ ID NO: 1; and a CDR set corresponding to a heavy chain having a variable domain comprising an amino acid sequence as set forth in SEQ ID NO: 2.

Complementarity determining regions (CDRs) are known as hypervariable regions both in the light chain and the heavy chain variable domains. The more highly conserved portions of variable domains are called the framework (FR). Complementarity determining regions (CDRs) and framework regions (FR) of a given antibody may be identified using the system described by Kabat et al. supra; Lefranc et al., supra and/or Honegger and Pluckthun, supra. Also familiar to those in the art is the numbering system described in Kabat et al. (1991, NIH Publication 91-3242, National Technical Information Service, Springfield, Va.). In this regard Kabat et al. defined a numbering system for variable domain sequences that is applicable to any antibody. One of ordinary skill in the art can unambiguously assign this system of “Kabat numbering” to any variable domain amino acid sequence, without reliance on any experimental data beyond the sequence itself.

### Transmembrane Domains

In addition to the extracellular region of a CAR which is responsible for binding the antigen, i.e., PSMA, a CAR comprises a transmembrane domain. A transmembrane domain of an anti-PSMA CAR of the present invention can be in any form known in the art, and as described below.

As used herein, the term “transmembrane domain” refers to any polypeptide structure that is thermodynamically stable in a cell membrane, preferably a eukaryotic cell membrane (e.g., a mammalian cell membrane).

Transmembrane domains compatible for use in the anti-PSMA CARs disclosed herein may be obtained from any natural transmembrane protein, or a fragment thereof. Alternatively, the transmembrane domain can be a synthetic, non-naturally occurring transmembrane protein, or a fragment thereof, e.g., a hydrophobic protein segment that is thermodynamically stable in a cell membrane (e.g., a mammalian cell membrane).

In some embodiments, the transmembrane domain is derived from a type I membrane protein, i.e., a membrane protein having a single membrane-spanning region that is oriented such that the N-terminus of the protein is present on the extracellular side of the lipid bilayer of the cell and the C-terminus of the protein is present on the cytoplasmic side. In some embodiments, the transmembrane protein may be derived from a type II membrane protein, i.e., a membrane protein having single membrane-spanning region that is oriented such that the C-terminus of the protein is present on the extracellular side of the lipid bilayer of the cell and the N-terminus of the protein is present on the cytoplasmic side. In yet other embodiments, the transmembrane domain is derived from a type III membrane protein, i.e., a membrane protein having multiple membrane-spanning segments.

Transmembrane domains for use in the anti-PSMA CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids in length. Examples of synthetic transmembrane domains are known in the art, for example in U.S. Pat. No. 7,052,906 B1 and PCT Publication No. WO 2000/032776 A2, the contents of which are herein incorporated by reference, and in particular, the disclosure regarding synthetic transmembrane domains).

In one embodiment, the anti-PSMA CAR comprises a trans membrane domain having the amino acid sequence of any one of SEQ ID NOs: 12, 13 or 18.

In some embodiments, the transmembrane domain of the anti-PSMA CAR comprises a transmembrane domain of CD3 zeta, or a functional portion thereof, such as a transmembrane domain that comprises the amino acid sequence LCYLLDGILFIYGVILTALFL (SEQ ID NO: 12), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 12.

In some embodiments, the transmembrane domain of the anti-PSMA CAR comprises a transmembrane domain of CD3 zeta, or a functional portion thereof, such as a transmembrane domain that comprises the amino acid sequence LDPKLCYLLDGILFIYGVILTALFLRVK (SEQ ID NO: 13), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 13.

In some embodiments, the transmembrane domain of the anti-PSMA CAR comprises a transmembrane domain of human CD28 (e.g., Accession No. P01747.1), or a functional portion thereof, such as a transmembrane domain that comprises the amino acid sequence FWVLVVVGGVLACYSLLVTVAFIIFWV (SEQ ID NO: 18), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 18.

In one embodiment, the transmembrane domain used in an anti-PSMA CAR is derived from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD40L/CD154, VEGFR2, FAS, and FGFR2B. In some embodiments, the transmembrane domain is derived from CD8α. In some embodiments, the transmembrane domain is derived from 4-1BB/CD137. In other embodiments, the transmembrane domain is derived from CD28 or CD34.

### Intracellular Domains

Often, CARs are referred to as being a certain generation, e.g., a “first” or “second” generation. The “generations” of CARs typically refer to the intracellular signaling domains. First-generation CARs include only CD3ζ as an intracellular signaling domain, whereas second-generation CARs include a costimulatory domain often derived from either CD28 or 4-1BB. Third-generation CARs include two costimulatory domains, such as CD28, 4-1BB, and other costimulatory molecules.

Anti-PSMA CARs disclosed herein for use in the methods of the invention comprise an intracellular signaling domain. A signaling domain is generally responsible for activation of at least one of the normal effector functions of the cell (e.g., an immune cell, e.g., a T cell) in which the anti-PSMA CAR is being expressed. The term “effector function” refers to a specialized function of a cell. For example, the effector function of a T cell may include a cytolytic activity or helper activity, including, for example, the secretion of cytokines. Thus, the term “signaling domain” refers to the portion of a protein which transduces the effector function signal and directs the cell to perform a specialized function. While usually the entire intracellular signaling domain can be employed, in many cases it is not necessary to use the entire chain or domain. Thus, to the extent that a truncated portion of the intracellular signaling domain is used, such truncated portion (or functional portion) may be used in place of the intact domain as long as it transduces the effector function signal.

Examples of intracellular signaling domains suitable for use in the anti-PSMA CARs disclosed herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

In a preferred embodiment, the anti-PSMA CAR used in the methods of the invention comprises a human CD3 zeta signaling region, or a functional portion thereof. In one embodiment, the human CD3 zeta signaling region comprises the amino acid sequence set forth in SEQ ID NO: 5, provided below, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 5

(SEQ ID NO: 5)
LDPKLCYLLDGILFIYGVILTALFLRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPR.

In one embodiment, the CD3 zeta signaling region, or a functional portion thereof, comprises the amino acid sequence LDPK (SEQ ID NO: 11), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 11. In one embodiment, the CD3 zeta signaling region, or a functional portion thereof, comprises the amino acid sequence LCYLLDGILFIYGVILTALFL (SEQ ID NO: 12), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 12. In one embodiment, the CD3 zeta signaling region, or a functional portion thereof, comprises the amino acid sequence LDPKLCYLLDGILFIYGVILTALFLRVK (SEQ ID NO: 13), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 13. In one embodiment, the CD3 zeta signaling region, or a functional portion thereof, comprises the amino acid sequence RVKFSRSADAPAYQQGQNQLYNELNLGRREEYDVLDKRRGRDPEMGGKPRRKN PQEGLYNELQKDKMAEAYSEIGMKGERRRGKGHDGLYQGLSTATKDTYDALHM QALPPR (SEQ ID NO: 14), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 14.

In a preferred embodiment, the anti-PSMA CAR used in the methods of the invention comprises a human CD28 signaling region, or a functional portion thereof. In one embodiment, the human CD28 signaling region comprises the amino acid sequence set forth in SEQ ID NO: 16, provided below, or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 16

(SEQ ID NO: 16)
KIEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAPP
RDFAAYRS.

In one embodiment, the CD28 signaling region, or a functional portion thereof, comprises the amino acid sequence RSKRSRLLHSDYMNMTPRRPGPTRKHYQPYAP PRDFAAYRS (SEQ ID NO: 15), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 15. In one embodiment, the CD28 signaling region, or a functional portion thereof, comprises the amino acid sequence KIEVMYPPPYLDNEKSNGTIIHVKGKHLCPSPLFPGPSKP (SEQ ID NO: 17), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 17. In one embodiment, the CD8 region, or a functional portion thereof, comprises the amino acid sequence FWVLVVVGGVLACYSLLVTVAFIIFWV (SEQ ID NO: 18), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 18. In one embodiment, the CD28 signaling region, or a functional portion thereof, comprises the amino acid sequence RSKRSRLLHSDY MNMTPRRPGPTRKHYQPYAPPRDFAAYRS (SEQ ID NO: 19), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 19.

Examples of signaling domains that may be included in the intracellular domain of anti-PSMA CARs of the present invention include, but are not limited to, the signaling domains of TCR zeta, FcR gamma, FcR beta, CD3 gamma, CD3 delta, CD3 epsilon, CD5, CD22, CD79a, CD79b, and CD66d. In some embodiments, an anti-PSMA CAR of the present invention comprises a signaling domain of human CD3ζ. In other embodiments, an anti-PSMA CAR comprises a signaling domain from human CD28. Functional fragments of the foregoing examples are also included in the invention. In some embodiments, multiple signaling domains (e.g., one, two, three, four or more) are included in the intracellular domain of an anti-PSMA CAR.

In some embodiments, the intracellular domain of an anti-PSMA CAR of the present invention further comprises a co-stimulatory signaling domain. In some embodiments, the intracellular domain of the anti-PSMA CAR of the present invention comprises a signaling domain and a co-stimulatory domain. The term “co-stimulatory signaling domain,” as used herein, refers to a portion of a protein that mediates signal transduction within a cell to induce a response, e.g., an effector function. The co-stimulatory signaling domain of an anti-PSMA CAR of the present invention can be a cytoplasmic signaling domain from a co-stimulatory protein, which transduces a signal and modulates responses mediated by immune cells (e.g., T cells or NK cells).

Examples of co-stimulatory signaling domains for use in the chimeric receptors can be the cytoplasmic signaling domain of co-stimulatory proteins, including, without limitation, members of the B7/CD28 family (e.g., B7-1/CD80, B7-2/CD86, B7-H1/PD-L1, B7-H2, R7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB ligand/TNFSF9, BAFF/BLyS/TNFSF13B, BAFF R/TNFRSF13C, CD27/TNFRSF7, CD27 ligand/TNFSF7, CD30/TNFRSF8, CD30 ligand/TNFSF8, CD40/TNFRSF5, CD40/TNFSF5, CD40 ligand/TNFSF5, DR3/TNFRSF25, GITR/TNFRSF18, GITR ligand/TNFSF18, HVEM/TNFRSF14, LIGHT/TNFSF14, lymphotoxin-alpha/TNF-beta, OX40/TNFRSF4, OX40 ligand/TNFSF4, RELT/TNFRSF19L, TACI/TNFRSF13B, TL1A/TNFSF15, TNF-α, and TNF RII/TNFRSF1B); members of the interleukin-1 receptor/toll-like receptor (TLR) superfamily (e.g., TLR1, TLR2, TLR3, TLR4, TLR5, TLR6, TLR7, TLR8, TLR9, and TLR10); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIR1, HLA Class I, HLA-DR, ikaros, integrin alpha 4/CD49d, integrin alpha 4 beta 1, integrin alpha 4 beta 7/LPAM-1, LAG-3, TCL1A, TCL1B, CRTAM, DAP10, DAP12, MYD88, TRIF, TIRAP, TRAF, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory domain comprises an intracellular domain of an activating receptor protein selected from the group consisting of α₄β₁ integrin, β₂ integrins (CD11a-CD18, CD11b-CD18, CD11b-CD18), CD226, CRTAM, CD27, NKp46, CD16, NKp30, NKp44, NKp80, NKG2D, KIR-S, CD100, CD94/NKG2C, CD94/NKG2E, NKG2D, PENS, CEACAM1, BY55, CRACC, Ly9, CD84, NTBA, 2B4, SAP, DAP10, DAP12, EAT2, FcRγ, CD3ζ, and ERT. In some embodiments, the co-stimulatory domain comprises an intracellular domain of an inhibitory receptor protein selected from the group consisting of KIR-L, LILRB1, CD94/NKG2A, KLRG-1, NKR-P1A, TIGIT, CEACAM, SIGLEC 3, SIGLEC 7, SIGLEC9, and LAIR-1.

In some embodiments, an anti-PSMA CAR comprises an intracellular domain comprising at least one co-stimulatory signaling domain selected from the group consisting of CD27, CD28, 4-1BB (CD137), OX40, CD30, CD40, PD1, ICOS, lymphocyte function-associated antigen-1 (LFA-1), CD2, CD7, LIGHT, NKG2C, and B7-H3.

In some embodiments, the anti-PSMA CAR comprises the intracellular domain of CD3 zeta, or a functional portion thereof. In some embodiments, the intracellular domain of CD3 zeta, or a functional portion thereof, comprises the amino acid sequence RVKFSRSADAPAYQQGQNQLYNELNLGRREEYDVLDKRRGRDPEMGGKPRRKN PQEGLYNELQKDKMAEAYSEIGMKGERRRGKGHDGLYQGLSTATKDTYDALHM QALPPR (SEQ ID NO: 14), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 14.

In some embodiments, the anti-PSMA CAR comprises the intracellular domain of CD28, or a functional portion thereof. In some embodiments, the intracellular domain of CD28, or a functional portion thereof, comprises the amino acid sequence RSKRSRLLHSDYMNMTPRRPGPTRKHYQPYAPPRDFAAYRS (SEQ ID NO: 15), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 15. In some embodiments, the intracellular domain of CD28, or a functional portion thereof, comprises the amino acid sequence RSKRSRLLHSDYMN MTPRRPGPTRKHYQPYAPPRDFAAYRS (SEQ ID NO: 19), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 19.

In some embodiments, the anti-PSMA CAR comprises the intracellular domain of 4-IBB, or a functional portion thereof. In some embodiments, the intracellular domain of 4-IBB, or a functional portion thereof, comprises the amino acid sequence KRGRKKLLYIFKQPFMRPVQTTQEEDGCSCRFPEEEEGGCEL (SEQ ID NO: 20), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 20.

In some embodiments, an anti-PSMA CAR of the present invention may comprise more than one co-stimulatory signaling domain (e.g., 2, 3, 4, 5, 6, 7, 8, or more co-stimulatory signaling domains). In some embodiments, the anti-PSMA CAR comprises two or more co-stimulatory signaling domains from different co-stimulatory proteins, such as any two or more co-stimulatory proteins described herein. In some embodiments, the anti-PSMA CAR comprises two or more co-stimulatory signaling domains from the same co-stimulatory protein (i.e., repeats).

Selection of the type(s) of co-stimulatory signaling domain(s) may be based on factors such as the type of host cell that will be expressing the anti-PSMA CAR (e.g., T cells, NK cells, macrophages, neutrophils, or eosinophils), and the desired cellular effector function (e.g., an immune effector function).

The signaling sequences (i.e., a signaling domain and/or a co-stimulatory signaling domain) in the intracellular domain may be linked to each other in a random or specified order. The intracellular domain of the anti-PSMA CAR may comprise one or more linkers disposed between the signaling sequences. In some embodiments, the linker may be a short oligo- or a polypeptide linker, e.g., between 2 and 10 amino acids (e.g., 2, 3, 4, 5, 6, 7, 8, 9, or 10 amino acids) in length. In some embodiment, the linker may be more than 10 amino acids in length. Any linker disclosed herein, or apparent to those of skill in the art, may be used in the intracellular domain of an anti-PSMA CAR of the present invention.

### Other

In some embodiments, the anti-PSMA CAR further comprises a hinge region. In some embodiments, the hinge region is located between the scFv antibody region and the transmembrane domain. A hinge region is an amino acid segment that is generally found between two domains of a protein and may allow for flexibility of the anti-PSMA CAR and movement of one or both of the domains relative to one another.

In some embodiments, the hinge region comprises from about 10 to about 100 amino acids, e.g., from about 15 to about 75 amino acids, from about 20 to about 50 amino acids, or from about 30 to about 60 amino acids. In some embodiments, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. In some embodiments the hinge region is more than 100 amino acids in length.

In some embodiments, the hinge region is a hinge region of a naturally-occurring protein. Hinge regions of any protein known in the art to comprise a hinge region may be used in the anti-PSMA CARs described herein. In some embodiments, the hinge region is at least a portion of a hinge region of a naturally occurring protein and confers flexibility to the extracellular region of the anti-PSMA CAR. In some embodiments, the hinge region is a CD8 hinge region. In some embodiments, the hinge region is a CD8a hinge region. In some embodiments, the hinge region is a portion of a CD8 hinge region, e.g., a fragment containing at least 15 (e.g., 20, 25, 30, 35, or 40) consecutive amino acids of the CD8 hinge region. In some embodiments, the hinge region is a portion of a CD8a hinge region, e.g., a fragment containing at least 15 (e.g., 20, 25, 30, 35, or 40) consecutive amino acids of the CD8a hinge region.

In some embodiments, a anti-PSMA CAR comprises the CD8 hinge region, or a functional portion thereof. In some embodiments, the CD8 hinge region, or a functional portion thereof, comprises the amino acid sequence KPTTTPAPRPPTPAPTIASQPLSLR PEACRPAAGGAVHTRGLDFA (SEQ ID NO: 4), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 4.

In some embodiments, the hinge region is a hinge region of an antibody (e.g., IgG, IgA, IgM, IgE, or IgD antibodies). In some embodiments, the hinge region is the hinge region that joins the constant domains CH1 and CH2 of an antibody. In some embodiments, the hinge region is of an antibody and comprises the hinge region of the antibody and one or more constant regions of the antibody. In some embodiments, the hinge region comprises the hinge region of an antibody and the CH3 constant region of the antibody. In some embodiments, the hinge region comprises the hinge region of an antibody and the CH2 and CH3 constant regions of the antibody.

In some embodiments, the hinge region is a non-naturally occurring peptide. In some embodiments, the hinge region is a (GlyₓSer)ₙ linker, wherein x and n, independently can be an integer between 3 and 12, including 3, 4, 5, 6, 7, 8, 9, 10, 11, 12, or more. In some embodiments, the hinge region is (Gly₄Ser)ₙ, wherein n can be an integer between 3 and 60, or more, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60. In some embodiment, the hinge region comprises glycine and serine repeats, such as (Gly₄Ser)ₙ, where n is a positive integer equal to or greater than 1 (SEQ ID NO: 31). In some embodiments, the hinge region is (Gly₄Ser)₃ (SEQ ID NO: 22). In some embodiments, the hinge region is (Gly₄Ser)₆ (SEQ ID NO: 24). In some embodiments, the hinge region is (Gly₄Ser)₉ (SEQ ID NO: 25). In some embodiments, the hinge region is (Gly₄Ser)₁₂ (SEQ ID NO: 26). In some embodiments, the hinge region is (Gly₄Ser)₁₅ (SEQ ID NO: 27). In some embodiments, the hinge region is (Gly₄Ser)₃₀ (SEQ ID NO: 28). In some embodiments, the hinge region is (Gly₄Ser)₄₅ (SEQ ID NO: 29). In some embodiments, the hinge region is (Gly₄Ser)₆₀ (SEQ ID NO: 30).

In some embodiments, the hinge region is an extended recombinant polypeptide (XTEN), which is an unstructured polypeptide consisting of hydrophilic residues of varying lengths (e.g., 10-80 amino acid residues). Amino acid sequences of XTEN peptides are known in the art (see, e.g., U.S. Pat. No. 8,673,860, the contents of which are herein incorporated by reference). In some embodiments, the hinge region is an XTEN peptide and comprises 60 amino acids. In some embodiments, the hinge region is an XTEN peptide and comprises 30 amino acids. In some embodiments, the hinge region is an XTEN peptide and comprises 45 amino acids. In some embodiments, the hinge region is an XTEN peptide and comprises 15 amino acids.

In some embodiments, the hinge region is a non-naturally occurring peptide. In some embodiments, the hinge region is disposed between the C-terminus of the scFv and the N-terminus of the transmembrane domain of the CAR.

In some embodiments, the CAR comprises a tag used for identification of the CAR. For example, an anti-PSMA CAR may include a V5 tag. The V5 epitope tag is derived from a small epitope (Pk) present on the P and V proteins of the paramyxovirus of simian virus 5 (SV5). The V5 tag is usually used with all 14 amino acids (GKPIPNPLLGLDST; SEQ ID NO: 3), although it has also been used with a shorter 9 amino acid sequence (IPNPLLGLD; SEQ ID NO: 9).

In some embodiments, the CAR comprises a signal peptide. Signal peptides facilitate the expression of the CAR of the cell surface. Signal peptides, including signal peptides of naturally occurring proteins or synthetic, non-naturally occurring signal peptides, that are compatible for use in the CARs described herein will be evident to those of skill in the art. In some embodiments, the signal peptide is disposed N-terminus of the antigen-binding portion of the CAR. In some embodiments, the signal peptide comprises the amino acid sequence MEWSWVFLFFLSVTTGVHS (SEQ ID NO: 10), or an amino acid sequence having at least 85%, 86%, 87%, 88%, 89%, 90%, 91%, 92%, 93%, 94%, 95%, 96%, 97%, 98%, 99% or more sequence identity to the amino acid sequence of SEQ ID NO: 10:

### Host Cells

The present invention includes administration of a population of host cells that express CARs, e.g., anti-PSMA CARs, described herein, or a population of host cells which are transduced with nucleic acid molecules encoding anti-PSMA CARs described herein. In some embodiments, the host cells are immune cells (e.g., T cells, NK cells, macrophages, monocytes, neutrophils, eosinophils, cytotoxic T lymphocytes, regulatory T cells, or any combination thereof). In some embodiments, the host cells are T cells. In some embodiments, the host cells are natural killer (NK) T cells or placental-derived NK cells.

In one embodiment, cells used in the invention are autologous cells. The term “autologous” refers to any material derived from the same individual to whom it is later to be re-introduced into the individual. Thus, in certain embodiment, the anti-PSMA CAR expressing cell is taken from a human subject having prostate cancer, transduced with a DNA vector encoding the anti-PSMA CAR, and re-introduced (e.g., infused) back into the subject for treatment.

A population of immune cells for use in the invention can be obtained from any source, such as peripheral blood mononuclear cells (PBMCs), bone marrow, tissues such as spleen, lymph node, thymus, or tumor tissue. A source suitable for obtaining the type of host cells desired would be evident to one of skill in the art. In some embodiments, the population of immune cells is derived from PBMCs.

A cell (e.g., a T cell or a Natural Killer (NK) cell) used herein is engineered to express an anti-PSMA CAR. To create the host cells that express an anti-PSMA CAR disclosed herein, expression vectors for stable or transient expression of the anti-PSMA CAR may be constructed via conventional methods and introduced into the isolated host cells. For example, nucleic acids (e.g., DNA or mRNA) encoding the anti-PSMA CAR may be cloned into a suitable expression vector, such as a viral vector in operable linkage to a suitable promoter. The expression vector may be provided to a cell in the form of a viral vector. Viral vector technology is well known in the art and is described, for example, in Sambrook et al. (2012) MOLECULAR CLONING: A LABORATORY MANUAL, volumes 1-4, Cold Spring Harbor Press, NY, and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., as disclosed in PCT Application Nos. WO 01/96584; WO 01/29058; and U.S. Pat. No. 6,326,193). Suitable vectors and methods for producing vectors containing transgenes are well known and available in the art. In some embodiments, the vector is a viral vector. In some embodiments the viral vector is selected from the group consisting of a retroviral vector, a lentiviral vector, an adenovirus vector, and an adeno-associated vector.

A variety of promoters can be used for expression of an anti-PSMA CAR described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, herpes simplex tk virus promoter. Additional promoters for expression of an anti-PSMA CAR include any constitutively active promoter in a mammalian cell (e.g., an immune cell). Alternatively, any regulatable promoter may be used, such that its expression can be modulated within a host cell.

Vectors for use in the present invention may contain, for example, one or more of the following: a selectable marker gene (e.g., a neomycin gene for selection of stable or transient transfectants); an enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the anti-PSMA CAR.

Methods of delivering nucleic acids encoding an anti-PSMA CAR (e.g., a vector) to a host cell are well known in the art. Nucleic acids encoding an anti-PSMA CAR (e.g., DNA or mRNA) can be introduced into host cells using any of a number of different methods, for instance, commercially available methods which include, but are not limited to, electroporation (Amaxa Nucleofector-II (Amaxa Biosystems), ECM 830 (BTX) (Harvard Instruments), or the Gene Pulser II (BioRad), Multiporator (Eppendorf), cationic liposome mediated transfection using lipofection, polymer encapsulation, peptide mediated transfection, or biolistic particle delivery systems such as “gene guns” (see, for example, Nishikawa et al. (2001) HUM GENE THER. 12(8): 861-70.

In some embodiments, vectors encoding an anti-PSMA CAR of the present invention are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984; and WO 95/00655).

Host cells included in the present invention may express more than one type of anti-PSMA CAR (e.g., two types of anti-PSMA CAR). The expression of more than one type of anti-PSMA CAR may be particularly advantageous for therapeutic purposes.

### Kits

The invention also provides kits comprising one or more compositions disclosed herein. Kits of the invention include one or more containers comprising a population of host cells comprising an anti-PSMA CAR disclosed herein, and in some embodiments, further comprise instructions for use in accordance with any of the methods described herein. The kit may further comprise a description of selection an individual suitable or treatment, e.g., a subject having cancer associated with PSMA expression. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, the kit comprises a) a composition comprising a population of host cells comprising an anti-PSMA CAR, wherein the anti-PSMA CAR comprises an anti-PSMA scFv, a transmembrane domain, and an intracellular signaling domain, and b) instructions for administering the population of host cells to a subject for the effective treatment of cancer. In some embodiments, said cancer is prostate cancer.

In one embodiment, the invention provides a kit comprising a population of host cells expressing anti-PSMA CARs. In some embodiments, the population of host cells comprising anti-PSMA CARs of the invention is comprised of from about 1×10¹ host cells to about 1×10¹² host cells. Alternatively, the population of host cells comprising anti-PSMA CARs include about 1×10² host cells to about 1×10¹² host cells; about 1×10³ host cells to about 1×10¹² host cells; about 1×10⁴ host cells to about 1×10¹² host cells; about 1×10⁵ host cells to about 1×10¹² host cells; about 1×10⁶ host cells to about 1×10¹² host cells; about 1×10⁷ host cells to about 1×10¹² host cells; about 1×10⁸ host cells to about 1×10¹² host cells; about 1×10⁹ host cells to about 1×10¹² host cells; about 1×10⁸ host cells to about 1×10¹¹ host cells; about 1×10⁸ host cells to about 1×10¹⁰ host cells; or about 1×10⁷ host cells to about 1×10¹⁰ host cells.

In other embodiments, the kit comprises a) a composition comprising a nucleic acid molecule encoding an anti-PSMA CAR, wherein the anti-PSMA CAR comprises an anti-PSMA scFv antibody, a transmembrane domain, and an intracellular signaling domain; and b) instructions for introducing the nucleic acid molecule encoding an anti-PSMA CAR into an isolated host cell.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information.

The instructions relating to the use of the compositions disclosed herein include information as to dosage, dosing schedule, and route of administration for the intended treatment. The containers may be unit doses, bulk packages (e.g., multi-dose packages) or sub-unit doses.

The present invention is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example

The designer T cell (dTc) approach is an innovation versus vaccines that bypasses immunization, and provides a high affinity receptor by engineering [7]. Often these receptors (chimeric antigen receptors or CARs) are fusions of antibody (Ab) binding domains with signaling chains of the T cell receptor (TCR). A version of this strategy was recently demonstrated to suppress and potentially cure CLL [8, 9].

A CAR was previously engineered to create an anti-prostate specific membrane antigen (PSMA) dTc that specifically target and kill prostate cancer in vitro and in in vivo models [10] (see Ma, Q, Safar M, Holmes E, et al. Anti-prostate specific membrane antigen designer T cells for prostate cancer therapy. Prostate 2004; 61:12-25). A schematic of the anti-PSMA CAR is provided in FIG. 7. Although this was a 1st generation (1st gen) zeta-only CAR, it has properties of proliferation with antigen contact as opposed to apoptosis/AICD seen with other 1st gen CARs in dTcs that were encouraging for a better therapeutic impact. IL2 has been previously shown to eradicate established tumors in animal models using 1st or 2nd gen dTc, demonstrating the importance of IL2 with TILs in human studies.

A Phase I clinical trial was devised and is described below. To enhance the survival of the infused dTc, a “hematopoietic space” was created with non-myeloablative (NMA) chemotherapy (“conditioning”) before T cell infusion. This strategy was shown of benefit with tumor-infiltrating lymphocytes (TILs) in melanoma, effectively increasing patient “drug exposure” via the increased numbers of TILs [11]. A T cell dose escalation was planned to achieve a minimum 20% engraftment of infused activated cells post marrow recovery. Low dose IL2 (LDI) was administered to sustain activation of the infused dTc.

Engraftments of 5-56% were measured, with T cell expansions of 20-600-fold after 2w. Plasma IL2 was at predicted levels in two subjects, but was as much as 20-fold below prediction with high engraftments wherein expanded numbers of activated T cells were thought to deplete IL2. Clinically, toxicities were acceptable, and clinical partial responses (PR) were obtained in 2/5 subjects. Unexpectedly, clinical response bore an inverse relationship with T cell engraftment (“drug exposure”) and a direct relationship with IL2 level. This was an hypothesis-generating observation suggesting higher IL2 is required to achieve the more profound clinical responses predicted with higher dTc exposures.

### Patients and Methods

Patients.

Patients with metastatic or recurrent prostate cancer and hormone-refractory (castrate-resistant) disease were enrolled in the study.

Vector.

GMP quality vector was prepared in collaboration with the National Gene Vector Lab, an NCRR resource. 1 mg of plasmid DNA for the anti-PSMA CAR [Ma et al, 2004a] was supplied to the NGVL. VPCs were re-generated with the PG13 cell line, 100 single cell clones generated, grown up and tested for titer on 293 and activated normal human T cells. The preferred clone was expanded into a master cell bank (MCB) and used for vector production, at 32 C with 24 hr harvests. 18 L of supernatant were obtained. The final titer was 2×10⁶/ml on 293 cells and 0.5×10⁶/m1 on activated T cells.

Dose Preparation.

Patients underwent leukopheresis for 3-5 h to collect a peripheral blood mononuclear cell (PBMC)-enriched fraction, yielding 2-12×10⁹ cells, of which 60% were typically T cells. Leukopaks were transported to the RWMC Gene Therapy Facility where 1-2×10⁹ PBMC were placed in AIM V medium with 5% human serum at 4×10⁶ cells/ml with 30-60 ng/ml anti-CD3 antibody OKT3 [Ortho], with excess cells cryostored for possible repeat modification. On day +2 post activation, cells underwent transduction (Td) by spinfection with 1:1 dilution of supernatant, 2 ml/10⁷ T cells/well of a 6-well plate [Beaudoin et al, 2007], two times on day +2 and one time on day +3. T cells were assessed for CAR expression (below) 48-72 h post Td. A minimum fraction of 10% was the specification for patient dosing. Cells were harvested when expansions met dose, and cryopreserved. When microbiologic safety tests returned, the dose was released for patient administration.

Treatment Plan.

Upon enrollment, patients underwent leukocyte collection and mononuclear cell isolation. T cells were activated, transduced with retrovirus expressing anti-PSMA CAR and expanded [10]. Initially planned dose levels were: 10⁹, 10¹⁰, and 10¹¹ T cells, with a target of ≧20% engraftment of the infused T cells. This study target was met after 5 patients and the study was closed with no 10¹¹ cell doses administered.

Non-myeloablative chemotherapy (CyFlu) consisted of inpatient cyclophosphamide 60 mg/kg/d (with mesna), d-8 to d-7 followed by outpatient fludarabine 25 mg/m2/d, d-6 to d-2. On day 0, patients were admitted for dTc administration (over 15-30 minutes) then started on outpatient low dose IL2 (LDI) [PROLEUKIN®, Novartis Corporation] by continuous intravenous infusion (civi) at 75,000 IU/kg/d for 4w. This low dose IL2 regimen was near the outpatient MTD for prolonged continuous exposures.

“Rescue Packs”.

Stem cells were collected for marrow rescue in case of aplasia post chemotherapy in this older, often irradiated patient population. To avoid Th2 bias of the dTc, G-CSF [Neupogen, Amgen] induction (10 ug/d sc×5 d) was instituted after T cell collection and a separate leukopheresis performed. Collection was continued until a minimum of 2×10⁶ CD34+ cells/kg were recovered. Cells were transported to the RWMC Stem Cell Lab, then processed and cryopreserved per standard methods. Infusion of backup stem cells was to be triggered by day 21 in the event of non-recovery of the absolute neutrophil count. No patient required rescue pack infusion.

Cytokine Evaluations.

Serum IL2 was assayed by ELISA (Invitrogen).

Flow Cytometry.

Designer T cell samples were assayed for transduction by two-color staining for CD3, CD4 or CD8 and V5 antibodies [Invitrogen].

dTc Pharmacokinetics.

Heparinized blood samples were assayed for dTc by flow cytometry as above.

Q-PCR pharmacokinetics.

At specified times, 5 mL whole blood (WB) samples were collected into heparin-coated or citrated BD vacutainer tubes (BD Biosciences). Genomic DNA was isolated from 200 uL sample using the AxyPrep blood miniprep kit (Axygen Biosciences) and eluted in 100 uL TE buffer. Because of interference from heparin in PCR reactions, heparin-containing samples were pretreated with heparinase (below) that was avoided in later subjects by using only citrated tubes for sample collection.

Real-time PCR was performed using the BioRad CFX96 PCR detection system (BioRad). Reactions contained 11 uL eluted sample, 14 uL Maxima SYBR Green/ROX qPCR Master Mix (Fermentas) and 0.75 uL each primer at 10 uM. Primers were designed using Primer-Select (DNAStar) specific for CARs anti-PSMA (5-aggctgaggatttgggagtt-3 (SEQ ID NO: 32)/5-agacgctccaggcttcacta-3 (SEQ ID NO: 33), 182-bp spanning the SD38 GS linker) and anti-CEA (5-gcaagcattaccagccctat-3 (SEQ ID NO: 34)/5-gttctggccctgctggta-3 (SEQ ID NO: 35), 91-bp spanning the chimeric CD28-CD3z region) and albumin to quantitate absolute white blood cell (WBC) numbers (5-accatgcttttcagctctgg-3 (SEQ ID NO: 36)/5-tctgcatggaaggtgaatgt-3 (SEQ ID NO: 37), 81-bp). Amplifications were at 95 C for 10 min, 40 cycles at 95 C for 15 s, 60C for 20 s and 72 C for 20 s. Fluorescence data were acquired at the 72C extension phase. Product specificity was confirmed by melt curve analysis and gel electrophoresis. Absolute CAR copies and WBC numbers were calculated from plasmid standard curves and expressed relative to the baseline prescreen (PS) collection point. See FIG. 6 for results.

Heparinase Treatment of Samples.

Heparin collection tubes contain heparin, a polymer of sulfated glycosaminoglycan carbohydrates which binds DNA and inhibits PCR by occupying polymerase binding sites. To remove heparin, 75 ul of sample was treated with 15 uL of Heparinase I Flavobacterium heparinum (Sigma) for 2 h at 37 C. Heparinase I was dissolved at 1 mg per mL in 20 mM Tris-HCl pH 7.5, 50 mM NaCl, 4 mM CaCl2 and 0.01% BSA. 11 uL of heparinase-treated DNA was used for Q-PCR.

Detection of Immune Reaction Against CAR on dTc.

Sera from patients collected at 1 to 6 months post-therapy were incubated with Jurkat or Jurkat CAR+ T cell line at 1:5 dilution for 45 min on ice. Cells were washed and then incubated with fluorescence-tagged goat-anti-human Ig, and evaluated by flow-cytometry. Positive controls included anti-CEA CAR+ Jurkat cells reacted with human CEA-Fc [Ma et al, 2004b], detected with the same secondary Ab to show secondary Ab detects human Fc reacting with CAR+ cells, and anti-PSMA CAR+ Jurkat cells reacted with anti-V5 Ab (mouse), detected with goat anti-mouse secondary Ab to show the expected profile for positive serum with this cell line for patients in this study.

### Results

### Patient Treatments

Between September 2008 and April 2010, six patients with metastatic prostate cancer and rising PSAs were enrolled with doses prepared (Table 1), of which five received treatment. The median age was 61 years (range 51-75) with a median time since diagnosis of recurrent or metastatic disease of 21 months (range 8-51). All patients received prior pelvic radiation and 5/6 failed androgen deprivation. (One patient requested study enrolment who had completed six months of adjuvant Lupron one year prior to presentation, but without subsequently having demonstrated hormone refractory status.)

TABLE 1
Patient Characteristics

| | |
|---|---|
| Patients (n) | 6 |
| Median age (yeards, range 51-75) | 61 years |
| ECOG performance status (n) | |
| 0 | 1 |
| 1 | 5 |
| Median time since diagnosis (months, range 8-51) | 21 months |
| Gleason score | |
| ≧1 | 5 |
| <7 | 1 |
| Disease location | |
| Bone | 2 |
| Soft tissue | 2 |
| Both | 1 |
| Previous therapy (patients) | |
| LHRH analogue | 6 |
| Androgen blockade | 6 |
| Ketoconazole | 3 |
| Chemotherapy | 3 |
| Radical prostatectomy | 3 |
| External Radiotherapy | 6 |
| Baseline pain scores | |
| 0 | 4 |
| ≧1 | 2 |

The treatment plan began with autologous cell collections for dTc preparation. A separate filgrastim mobilization and leukopheresis for preparation of “rescue packs” in the event of excess marrow toxicity in this prostate cancer population that is typically older and of which some also receive pelvic irradiation. The separate collection for dTc manufacturing was to avoid the Th2 bias induction by G-CSF that could hamper the cytotoxic function of the derived dTc. Non-myeloablative (NMA) chemotherapy was initiated at day-8 with two days of cyclophosphamide followed by five days of fludarabine. After one day rest to allow for fludarabine clearance, cells were administered on day 0, with concurrent initiation of 28 d of IL2 by continuous intravenous infusion (civi) via central line. The treatment was entirely outpatient except for the two days of Cy for Mesna administration, and on the day of dTc administration for overnight observation.

The study had a Phase I dose escalation design to assess tolerability of anti-PSMA dTc with a target of 3 patients with 20% or greater engraftment of infused T cells post infusion. If no dose-limiting toxicities were encountered, this target engraftment was considered the optimum biologic “exposure,” indicating a highly successful insertion of cellular product into the lymphoid compartment. The dose yielding this engraftment would define the optimum biologic dose. Engraftments were unexpectedly vigorous (below), and this target was achieved with just 5 patients under the escalation plan (Table 2A, Dose and Engraftment), leading to study conclusion.

TABLE 2
dTc Treatment Data

| | A. Dose and engraftment | | | | | | | C. Response | | |
|---|---|---|---|---|---|---|---|---|---|---|
| | | | | | Total | | B. Interleukin 2 | PSA | | |
| | Dose | Dose Td | Blood dTc | Engrafted | engrafted | Fold | Peak plasma | change | PSA | |
| Patient | (cells) | fraction (%) | @2 w (%) | aTc (%) | aTc | increase | IL2 week 0-1 | (%) | delay | Overall |
| 1 | 10⁹ | 52 | 2.5 | 5 | 4.8E+10 | 48 | 2,300 | −50 | 78 d | PR |
| 2 | 10⁹ | 61 | 7.3 | 12 | 1.2E+11 | 120 | 2,100 | −70 | 150 d | PR |
| 3 | 10⁹ | 40 | 22.3 | 56 | 5.6E+11 | 560 | 200 | — | — | NR |
| 4 | 10¹⁰ | 40 | 20.6 | 52 | 5.2E+11 | 52 | 100 | — | — | NR |
| 5 | 10¹⁰ | 29 | 5.7 | 20 | 2.0E+11 | 20 | 600 | — | 25 d | mR |

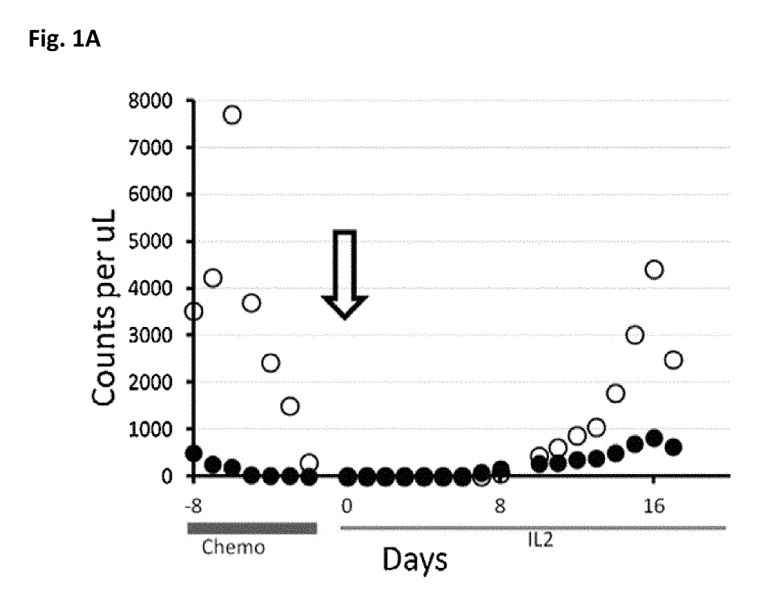

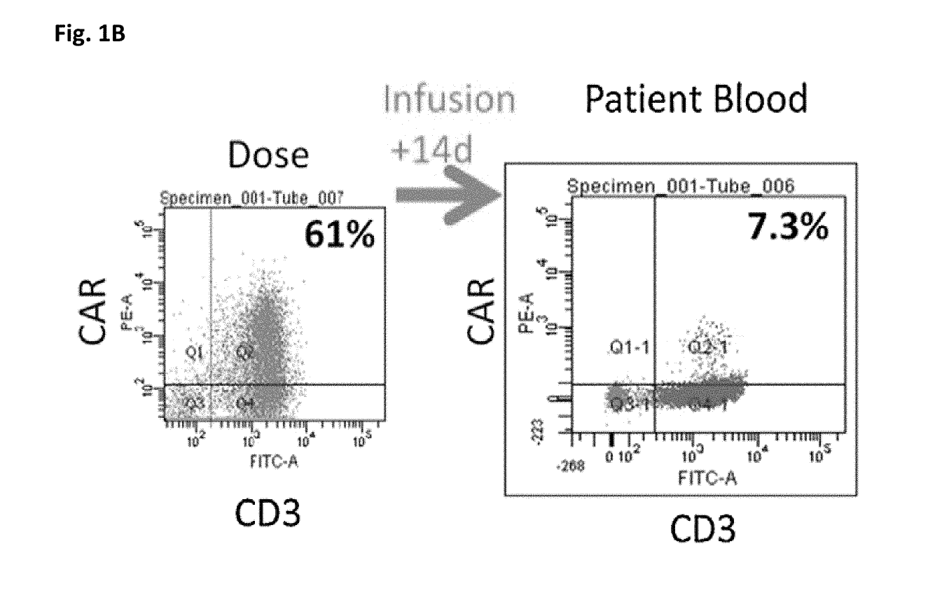

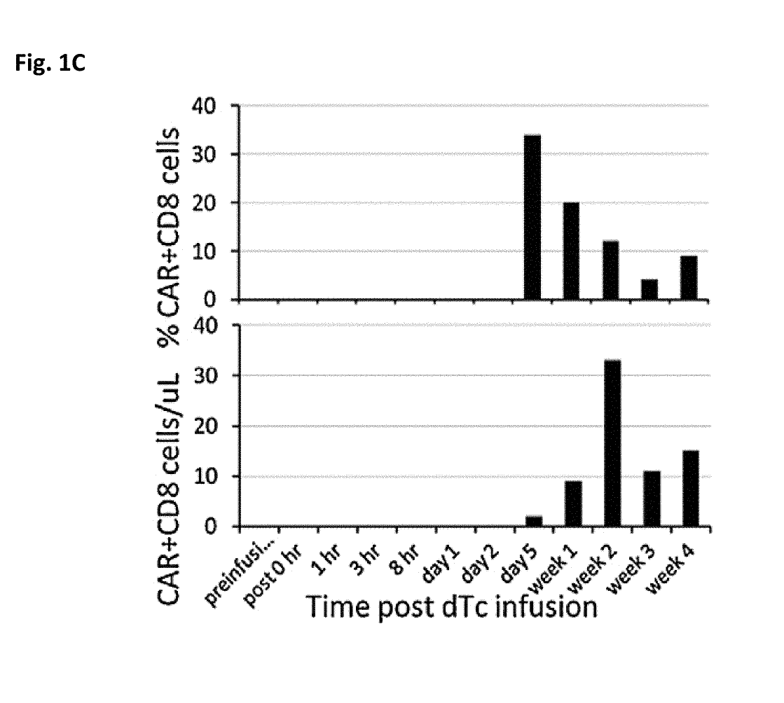

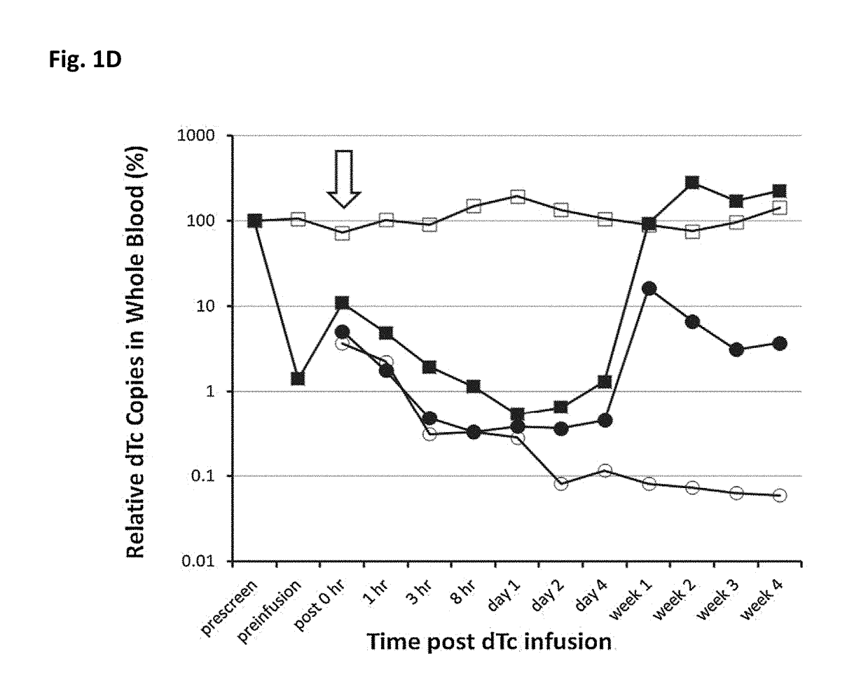

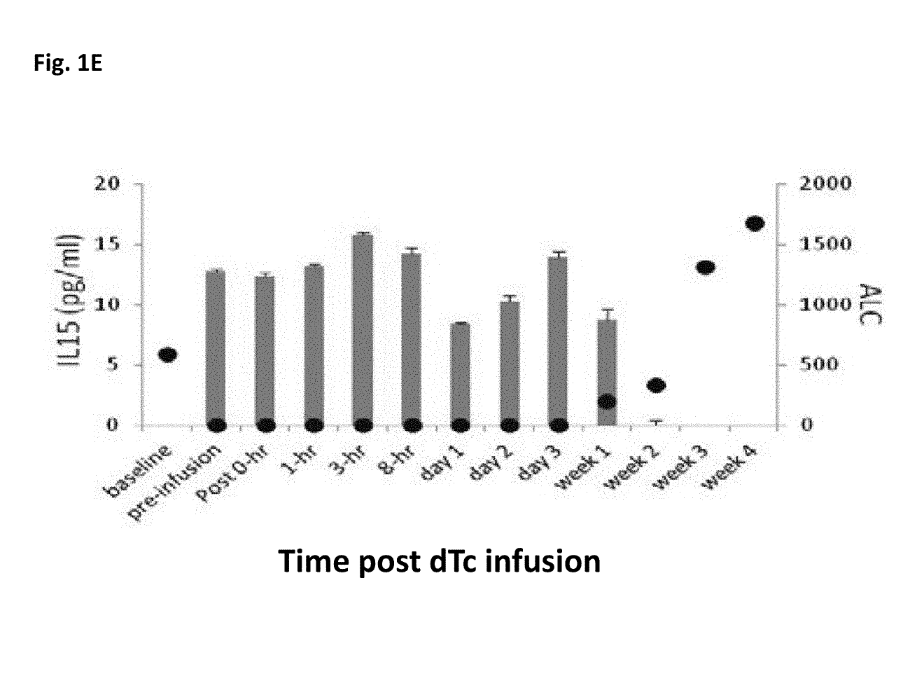

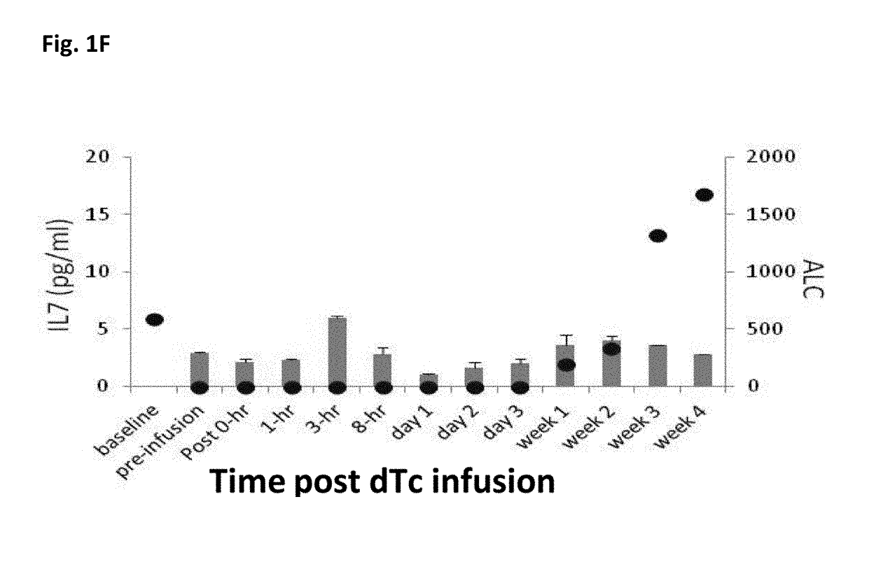

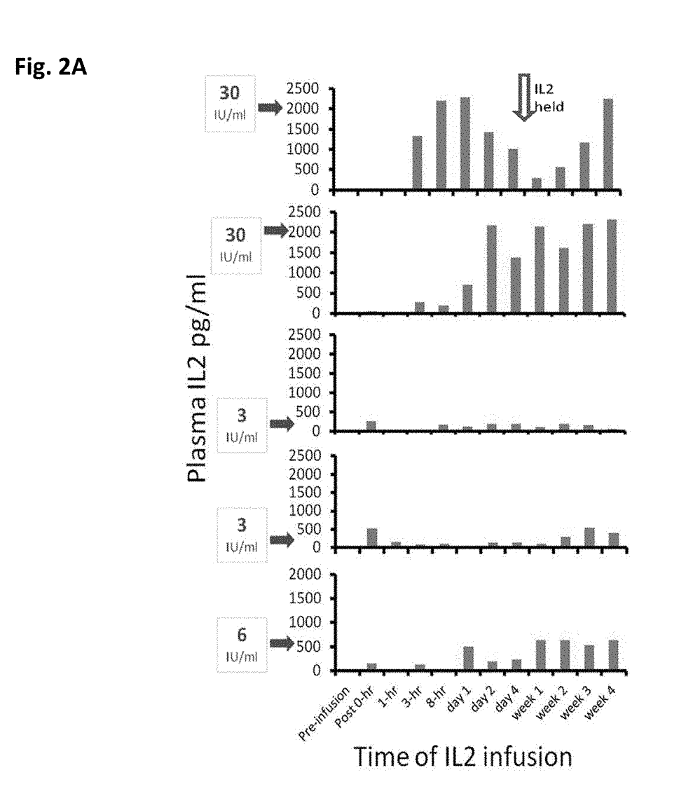

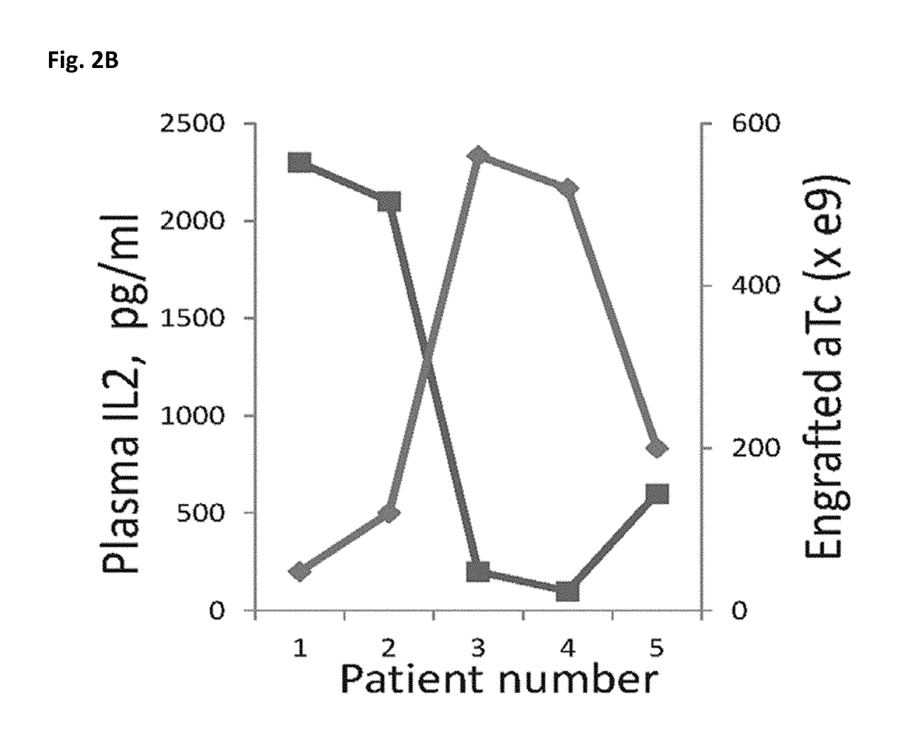

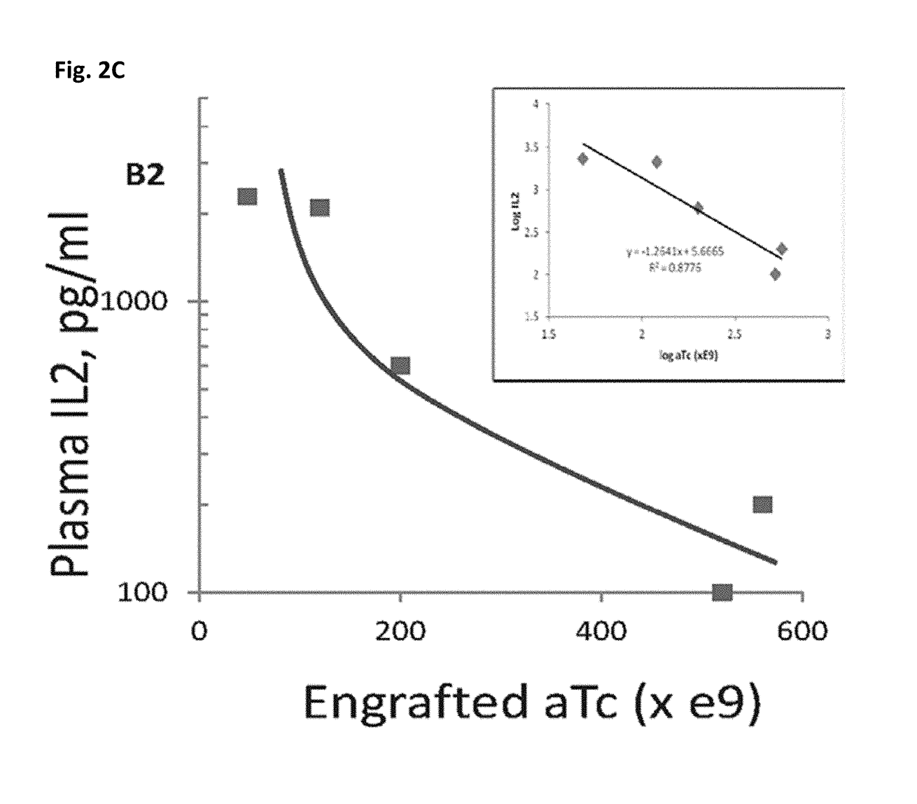

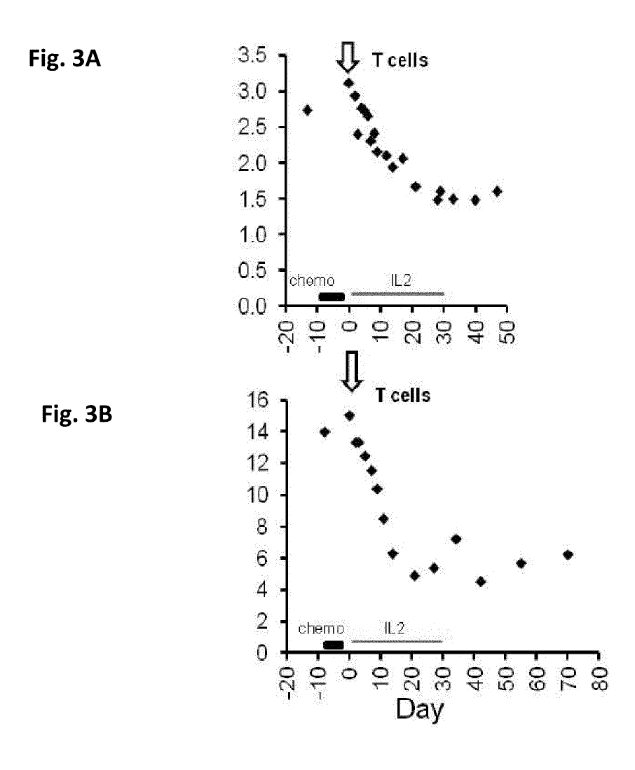

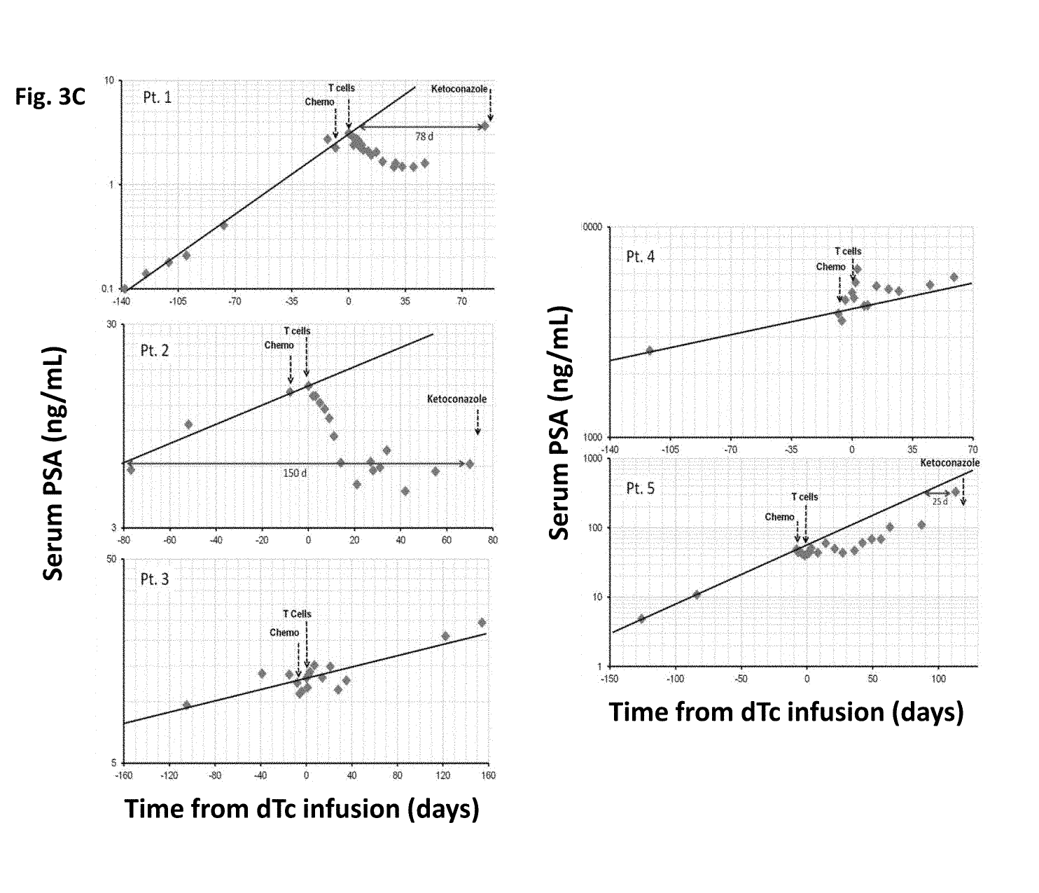

Table 2A. Dose and Engraftment. Dose transduced (Td) fraction and % dTc in blood at 2 w determined as in FIG. 1B. Engrafted activated T cells (aTc) as percent of total T cells estimated as ratio of % dTc at 2 w/dose % Td. Fold increase is total engrafted aTc/dose. For a fully reconstituted hematopoietic space. we apply a nominal total of 10¹² T cells in marrow, spleen, liver, lymph nodes, gut and blood as derived in note 2. The total engrafted aTc is estimated as the % engraftment×10¹² total T cells. Table 2B. Interleukin 2. Peak IL2 levels during first week from FIG. 2A. Table 2C. Response. PSA change and PSA delay from FIG. 3. Overall: PR, partial response; mR, minor “biologic” response; NR, no response.

### Pharmacokinetics

### Designer T Cells

The purpose of conditioning is to foster dTc engraftment and expansion. FIG. 1 shows the clinical profile of Pt2 at 10⁹ dose level. White cell counts declined rapidly during chemotherapy, with absolute leukopenia on day 0 at time of dTc infusion (FIG. 1A). ANC recovered to ≧500/ul by d10 (range all subjects d8-13) and ALC to >80% of baseline by d11 (range d10-15). From FIG. 1B, the original infused T cells were 61% CAR+ (“Dose”). On d14, when patients typically recovered their endogenous lymphocytes, the blood dTc fraction was 7.3% of total T cells. Allowing for original dose being <100% modified, engraftment efficiency of 7.3/61=12% is derived on d14, in which infused activated unmodified T cells also engrafted.

Engraftment was confirmed in all subjects, with 2.5-22% of circulating T cells being dTc after reconstitution at 2 weeks, corresponding to engraftment efficiencies of 5-56% (Table 2A). Whereas Pt 1 & 2 at the 10⁹ dose level had total engraftment fractions of 5-12%, Pt3, also at 10⁹ engrafted to 56%. Pt 4 & 5 with 10¹⁰ cell doses engrafted to 52% and 20%, respectively. Three patients achieved engraftments of ≧20%, one from dose level 1 and two from dose level 2, fulfilling accrual goals. These values are estimated to correspond to 5×10¹⁰ to >5×10¹¹ engrafted T cells post-infusion, representing expansions of 20-fold to nearly 600-fold (see Table 2A). The relative expansions were lower with the higher doses, as might be expected with an upper limit that reconstitution can achieve, i.e., normal T cells ˜1000/ul in blood and ˜10¹² whole body.

Kinetics of engraftment were assessed by flow. On the first day that cells were sufficient to analyze (wbc=0.2 on d5), CAR+ cells were at their highest percentage, and declined thereafter as endogenous T cells recovered post chemotherapy (FIG. 1C upper). The peak absolute number of CAR+ cells was at d14, with a leveling off at lower total levels that were stable by d21 through the end of the study period on d28 (FIG. 1C lower). This pattern was typical for all patients.

Conditioning did not impact initial pharmacokinetics but pharmacodynamics (engraftment) was strikingly altered. FIG. 1D compares two different patients by PCR with similar-size dTc doses, with and without conditioning. From the first point immediately post infusion (time=0 h) until 8 h, both settings showed similar initial pharmacokinetics, with a rapid 10-fold loss of dTc in circulation as cells distributed between blood and tissues. Subsequently, the simple infusion continued a further 5-fold decline from 8 h to d7 (50-fold decline overall), after which numbers were relatively stable for the duration of the month. In contrast, the patient with prior conditioning maintained cell numbers in blood from the 8 h time-point until d4, after which cells in blood expanded in a burst to yield a 50-fold increase by d7. Comparison of dTc levels in the blood at d14 showed a near 200-fold advantage of the conditioning. This pattern was evident across all patients.

IL7 and IL15 (but not IL2) have been reputed to drive T cell recovery after lymphopenic conditioning [12, 13]. Of note is that IL2 is considered neither necessary nor sufficient to foster engraftment. The same IL2 regimen had previously been applied in a prior CEA clinical trial [Junghans et al, 2001] and no engraftment was noted, nor was engraftment noted in the TIL studies of Rosenberg with high-dose IL2 co-administration [Rosenberg et al, 1994]. Murine studies show that engraftment does not require IL2 [Bracci et al, 2007]. Instead, the intention of IL2 in this study was to support the activated state of the T cells to sustain their cytotoxic activity in vivo.

Notably, IL15 was zero at baseline in all subjects, elevated with lymphodepletion at time of dTc infusion, then returning to baseline as ALC increased to normal, as shown in FIG. 1E. IL7 in the same subject began as unmeasurable, but did not decline post recovery as shown in FIG. 1F. In general, IL7 did not present a consistent pattern, in some cases non-zero at start, with minimal increase after conditioning and, in some cases peaking after lymphoid reconstitution.

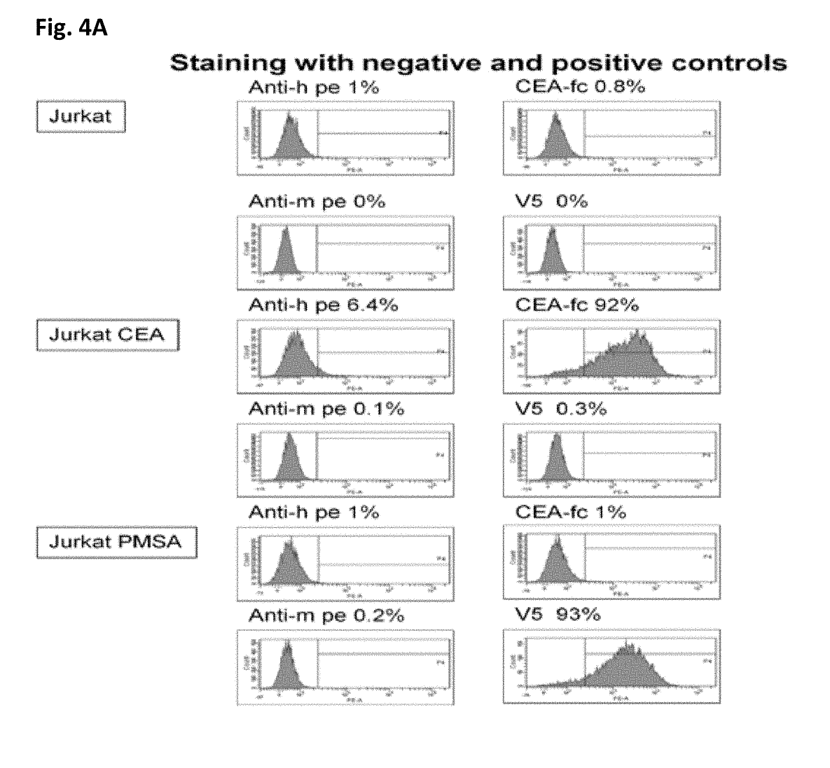

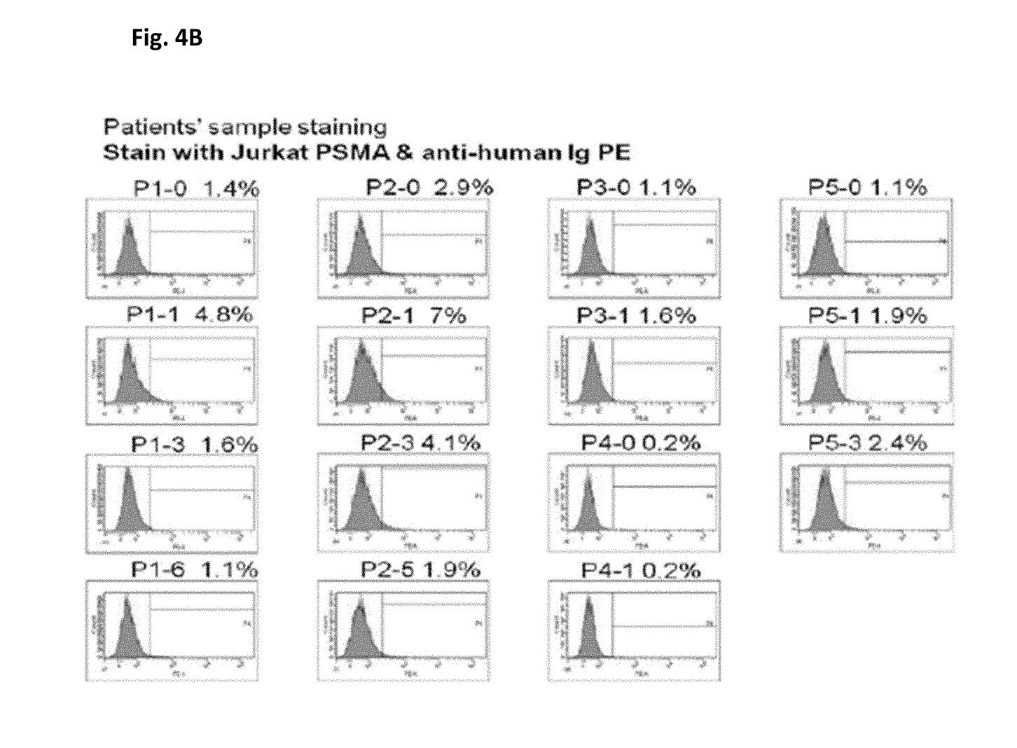

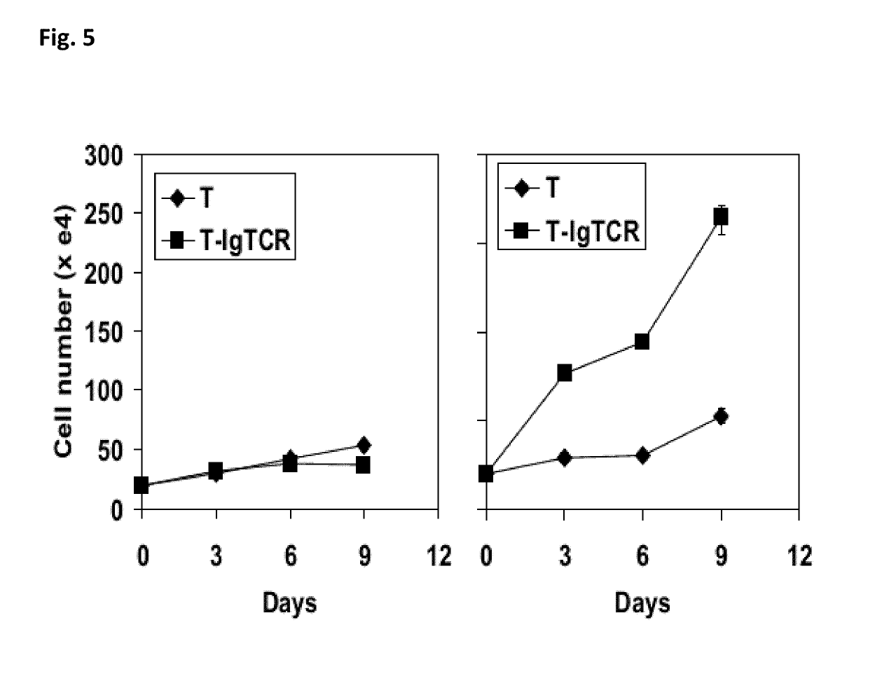

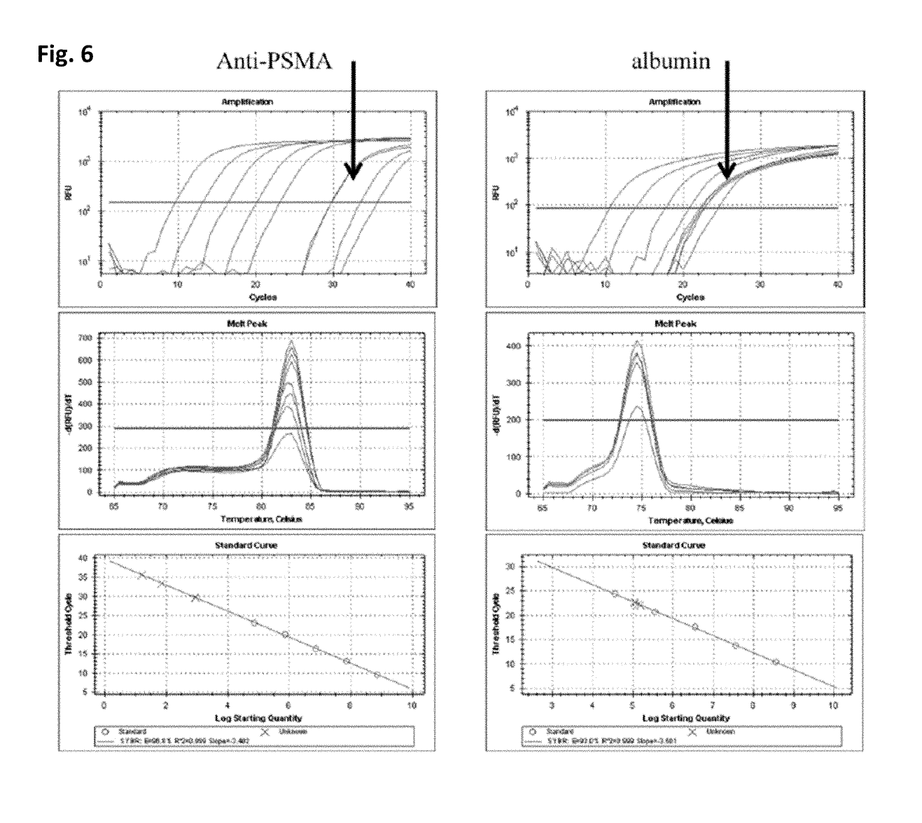

At times 1 to 6 months after dTc injection, sera were screened for reactivity against CAR+ T cells. No anti-CAR immune response was detected in any subject after treatment, as shown in FIG. 4.

### Interluekin 2

Because IL2 was considered a key component to success of the intervention during the original study design, blood IL2 was monitored to ensure adequate levels were obtained. Under the planned regimen, blood levels are predicted in the range of 1900+/−600 pg/ml (−30 IU/ml) [14]. When patient IL2 profiles were analyzed, however, striking differences were noted (FIG. 2A, Table 2B, Interleukin 2): Pts 1 & 2 both achieved high plasma IL2 (>2000 pg/ml) within days after initiation of therapy, whereas Pts 3 & 4 had much lower peak IL2 (100-200 pg/ml) during the critical first week of therapy, with an intermediate peak value (600 pg/ml) in Pt 5 (FIG. 2A). The high levels of Pts 1 & 2 are in the predicted range, whereas the low values are far below expectation. (Without IL2 co-administration, IL2 is undetectable in plasma even with very high dTc doses.)

Importantly, the observed blood levels of 100-2000+ pg/ml (1.5-35 IU/ml) span a critical range, with high levels sufficient to sustain T cell activity and low levels likely subtherapeutic, particularly for T cells in tissues where their action is required.

NOTE: From Konrad et al (1990), 1 MIU/m2/6 h (4 MIU/m2/d) by civi yields a steady state blood level of 39.2±13.8 IU/ml. The dosing herein is expressed per kg, 75 kiu/kg/d. For Patients 1 and 2, dosing was converted to BSA units and then calculated as expected values (±standard deviation) from data of Konrad et al.:

TABLE 3
Predicted IL2 levels in pg/ml.

| | | total | | | plasma IL2 (pg/ml) | | |
|---|---|---|---|---|---|---|---|
| | weight | IL2/d | BSA | IL2/bsa | predicted | | Observed |
| Pt # | (kg) | (mcg) | (m2) | (mcg/m2) | mean | SD | Value |
| 1 | 89.3 | 409 | 2.20 | 186 | 1823 | 642 | 2300* |
| 2 | 92.4 | 424 | 2.19 | 193 | 1895 | 667 | 2100** |

*p > 0.4;
**p > 0.8

TABLE 4
Predicted IL2 levels in IU.

| | | total | | | plasma IL2 (IU/ml) | | |
|---|---|---|---|---|---|---|---|
| | weight | IL2/d | BSA | IL2/bsa | predicted | | observed |
| Pt # | (kg) | (MIU) | (m2) | (MIU/m2) | mean | SD | value |
| 1 | 89.3 | 6.70 | 2.20 | 3.04 | 29.8 | 10.5 | 37.6* |
| 2 | 92.4 | 6.93 | 2.19 | 3.16 | 31.0 | 10.9 | 34.4** |

*p > 0.4;
**p > 0.8

The measured values for Patients 1 and 2 from Table 2B are 2300 pg/ml and 2100 pg/ml. Based on the potency standard for Proleukin of 18 MIU/1.1 mg, these values correspond to 37.6 IU/ml and 34.4 IU/ml, respectively. Thus, the measured IL2 peak values for Pts 1 & 2 are within the range of prediction, and those for Pts 3-5 (100 to 600 pg/ml; 1.6 to 9.8 IU/ml) are well below range.

Noted on the axes of the graphs in FIG. 2 A are positions of the unit measurements (IU/ml). 1 BRMP Unit of IL2 was defined as that which generates half-maximal proliferation of an IL2-dependent cell line, CTLL-2. The International Unit applied by Novartis is roughly ⅙^{th} of a BRMP unit for stimulatory activity [Hank et al, 1999]. That is, with 30 IU/ml, we are 5-fold above the ½ maximal stimulation dose, whereas with 1-6 IU/ml, we are at or below the V₂ stimulation dose. It is likely that these levels are still lower in tissues, and what is borderline in the plasma may be frankly deficient in tumor where activation needs to be maintained. Therefore, it is a reasonable speculation that low IL2 hampered dTc effectiveness, efficacy being seen only with high IL2.

### Pharmacodynamics

### IL2 Insufficiency and Activated T Cells

Causes of IL2 differences were examined. Repeat testing ruled out measurement artifacts, and mixing studies ruled out an inhibitor. Further, drug lot bioactivities, drug delivery and patient differences in terms catabolism were eliminated as sources of differences. With assay, drug and patient differences removed as causes, attention turned to the sole remaining component: the T cells themselves. It was hypothesized that engrafted activated T cells (aTc) consumed IL2 to mediate IL2 depletion, as explored in FIG. 2B. All cells in the dose, transduced (dTc) and untransduced T cells alike, are activated by anti-CD3 Ab prior to vector exposure, expressing IL2 receptors (IL2R), and engraft systemically and also bind IL2.

IL2 receptor (IL2R) rises to extremely high levels (up to 100,000/cell) in the post-activation period in which the complexity of low, intermediate and high affinity receptors change with time to fulfill different roles, then progressively decline over the ensuing days and weeks [Jacques et al, 1987]. The expansion of aTc post-infusion may be paralleled by the decline in binding sites/cell to maintain a steady “sink” for IL2 that yields relatively steady low plasma levels with net high engraftments through the monitoring period. It may be that an eventual high engrafted fraction at two weeks is paralleled by a high expansion rate in the first days with high-IL2R+ cells. This then gives the result that the IL2 steady state (plateau) in the first 1-2 days is already low and comparable to that seen at later times (e.g., day 14). (see calculations below)).

When this analysis was performed, a remarkable result obtained: peak IL2 in the critical 0-1 week period varied inversely with engraftment fraction: viewing Pts 1-5 in sequence (Table 2AB; FIG. 2B), IL2 was high with lowest engrafted fractions (5-12%); IL2 dropped to low with highest engraftments (>50%); then IL2 rose to intermediate with middle engraftment (20%). When plotted as IL2 versus engrafted fraction, the inverse relation was explicit and significant (p<0.01) (FIG. 2C). These data are consistent with IL2 depletion by aTc that engraft to high levels, with calculations supporting the plausibility of this scenario.

Calculations:

A 10% engraftment or 10¹¹ T cells (assuming total 10¹² T cells in an adult; Table 2A) with 1000 IL2R per cell (170 pmoles) could bind 3 ug of IL2. Assuming a distribution volume of 8 L for IL2 [Konrad et al, 1990], and a nominal IL2 level of 2000 pg/ml under our infusion protocol (without IL2 binding by aTc), a total body level of 16 ug IL2 is estimated at steady state. Binding of 3 ug of IL2 would lead to 3/16 or −20% depletion, or a ˜400 pg/ml reduction. Correspondingly, if engraftment were 50%, depletion of IL2 could be ˜15/16 or 94% depletion, to 100 pg/ml. 1% as many cells at earlier times post-infusion with 100-fold the IL2R would have the same binding capacity. Depending on actual levels of engraftment, IL2R levels, IL2 internalization rates, PK parameters and catabolic rates for IL2, the total of 10 ug IL2 per hour that is infused under our protocol could be reduced by 50% or 90% or more and generate these hindering effects for the infused dTc. There are many undetermined variables in this estimate but calculations demonstrate it is within the range of plausibility.

### Toxicity

Toxicities were assessed from chemotherapy, from IL2 and from the dTc themselves. From chemotherapy, major (grade 3/4) toxicities were hematologic, as expected: neutropenia and neutropenic fever (5/5 patients) and thrombocytopenia (3/5 patients), as described in Table 5. Neutropenic fever patients were admitted and administered iv antibiotics until defervescence and neutrophil recovery, according to hospital protocols. One patient required an appendectomy during neutropenia. All patients recovered ANC>500 within 14 days, and no patient required stem cell rescue. Toxicities attributed to IL2 were grade 1-2 fatigue, intermittent low-grade fevers, and myalgias. One patient had IL2 discontinued after 3 weeks for grade 2 skin rash. No toxicities were attributed to dTc targeting of normal tissues expressing PSMA (e.g., kidney, brain; see Discussion below). Notably, no “cytokine storm” was observed as previously documented in leukemia studies [8, 9, 15, 16], and cytokines correlating with such activity (IL6, TNF-alpha, interferon-gamma) were uniformly non-elevated by Kochendorfer et al [15] criteria (e.g., <100 pg/ml).

TABLE 5
Major Grade 3/4 Toxicities

| Toxicity | Patients (%) | |
|---|---|---|
| Neutropenia | 5 | (100) |
| Neutropenic fever | 5 | (100) |
| Thrombocytopenia | 3 | (60) |
| Anemia | 1 | (20) |
| zHypocalcemia | 1 | (20) |
| Hypophosphatemia | 1 | (20) |
| Appendicitis | 1 | (20) |

Of patients with neutropenic fevers, 3/5 had no identifiable source, one patient had a Streptococcus parasanguinis bacteremia along with Enterococcus faecalis urinary tract infection and one patient had Streptococcus viridians bacteremia. All were admitted to the hospital and treated with broad spectrum antibiotics with successful recovery. One patient developed acute appendicitis during week 4 of therapy requiring laparoscopic appendectomy and had an uneventful recovery. One patient developed a peripheral eosinophilia to 51% in the absence of respiratory symptoms or pulmonary findings on chest x-ray; the eosinophilia resolved upon completion of IL2 infusion.

### Response

Although only a Phase I study to test safety and engraftment, clinical responses were noted. PSA profiles are shown for Pts 1 & 2 (FIGS. 3A and 3B). During the conditioning period (d-8 to d0), the PSA continued its rise, showing, as expected, no net impact of chemotherapy by time of T cell infusion.

In these two patients, PSA fell promptly after dTc infusion, declining by 50% and 70% at their nadirs over the ensuing 1-2 months, meeting criteria of PR for prostate cancer (Table 2C, Response). After this, the patients' PSAs resumed their upward trajectories. No other patient met criteria for clinical response. We also examined PSA delay as a measure of benefit, as this has been proposed in other immune therapies as a survival surrogate [17-21]. PSA delays of 78 and 150 days were estimated for Pts 1 & 2 (FIG. 3B). Pts 3 & 4 did not deviate appreciably from the PSA projection and no PSA delay was estimated. Pt5 experienced a PSA persistently below projection, referred to herein as a “biologic” minor response (mR), with a PSA delay estimated as 25 days. (The term “biologic response” is used to refer to marker changes that indicate immune action against tumor, not meeting conventional response criteria.)

Pt1 lacked radiologic evidence of disease. Pt2 had a positive bone scan that was read at one month post dTc as showing stability or improvement (one lesion). Pts 3-5 without objective PSA declines had no follow-up scans.

NOTE: Cy is poorly active in prostate cancer: tested as a single agent, it produced only 1 PR in 48 subjects [Chlebowski et al., 1978; Muss et al., 1981; Saxman et al., 1992]. Nevertheless, to separate as far as possible chemotherapy effects from the dTc infusions, the Cy portion was placed at the front of the conditioning (d-8 to d-7) and completed a full week before dTc infusion (d0), reasoning that any anti-tumor activity of the drug would be manifest by this time. In all 5 subjects, however, the PSA stayed on its pre-conditioning trajectory without evidence of a chemo-effect. Fludarabine is an anti-metabolite that is highly specific for lymphoid cells and their malignancies; no impact on solid tumors would be expected. Finally, the observed response rate of 2 PR/5 subjects in this study was inconsistent with response due to Cy (1 PR/48) (p=0.02; Fisher exact test), suggesting that the observed response is dTc derived.

Correlates of Response/Non-Response

Looking for patient differences to explain responses/non-responses, nothing was suggestive in performance status, age, body habitus, disease status or treatment history.

When response was judged versus T cell dose, no relation to dose level was evident (p=0.6; Table 6A, Response vs Dose size). But when response was judged versus engraftment, the relation now approached significance (p=0.06; Table 6B, Response vs Engraftment)—yet in a direction opposite of expectation: more engraftment leading to less response. This pattern is un-typical of oncology drug responses: higher doses typically yield higher responses, but which may be constrained by increased toxicity in parallel. (Toxicity was not a factor with our dTc.) When response was considered versus IL2, the relationship was direct and significant (p=0.03), suggesting deficiency of IL2 was limiting the potential of higher exposures of dTc to mediate antitumor potency in vivo.

TABLE 6
Testing Correlates of Response

| | Fisher matrix | | | |
|---|---|---|---|---|
| | Response | | | |
| Correlated variables | NR | mR | PR | P-value |
| A. Response versus dose size | | | | |
| Dose level | | | | |
| Low | 1 | 0 | 2 | 0.6 |
| High | 1 | 1 | 0 | |
| B. Response versus | | | | |
| Engraft level | | | | |
| Low | 0 | 0 | 2 | 0.06 |
| Med | 0 | 1 | 0 | |
| High | 2 | 0 | 0 | |
| C. Response versus IL2 | | | | |
| IL2 level | | | | |
| Low | 2 | 0 | 0 | 0.03 |
| Med | 0 | 1 | 0 | |
| High | 0 | 0 | 2 | |

Response data from Table 2C. Table 6A. Correlates of “response versus dose size” tested by Fisher exact test, two-sided (H1: high dose induces more response or low dose induces more response; H0, response unrelated to dose). Dose level: low=1e9; high=1e10. Table 6B. Correlates of “response versus engraftment” by Fisher exact test, two-sided (H1: high engraftment induces more response or low engraftment induces more response; H0: response unrelated to engraftment). Engraftment level: low=<15%; medium=20%; high=>40%. Table 6C. Correlates of “response versus IL2” by Fisher exact test, single-sided (H1: more IL2 induces more response [if there is an IL2 effect, there is no biologic basis for low IL2 giving more response, hence test is appropriately single-sided]; H0: response unrelated to IL2 level). Using peak IL2 week 0-1 (Table 2B) as indicator: low <300; medium 400-800; high >1500.

Once 3 patients had been safely treated at the pre-specified optimum biologic “exposure” and the relation of high engraftments to low IL2 was established (p<0.01), with its predictably negative impact on dTc efficacy, it became problematic ethically to justify enrolling additional patients at the higher dTc doses as originally planned. That is, the optimal therapy seemed to require not merely an optimum biologic dose of dTc, which we had achieved by our definition, but also a matching optimum biologic dose of IL2 that is regulated by the pharmacodynamics of their interaction. This study was then terminated, as described below.

### Tests for Authenticity of IL2 Levels

A number of analyses were performed that confirmed the faithful delivery of drug, rule-out of inhibitor and other potential confounders, ultimately supporting that IL2 differences were authentic. The following analyses were conducted to determine if there was a flaw or confounding factor in this conclusion of IL2 differences:
1. Repetition of studies together: IL2 levels had been assayed sequentially and batch-wise for each patient after the one-month collection point. We then ran all samples together with all patients in the same assay. Identical results were obtained. This ruled out variability in the assay performance.
2. Mixing studies, to detect inhibitor that masks true IL2 levels: Patient sera with low IL2 were added to patient sera with high IL2 and the ELISA repeated. No suppression was of the high IL2 was observed. This ruled out an inhibitor substance such as high levels of soluble IL2 receptor (sCD25) or anti-IL2 Ab that could interfere with assay and underestimate IL2 present.
3. Hospital pharmacy assessment: Dose calculations were checked for all patients. Records confirmed IL2 cassettes were changed weekly, and residual pump volumes indicated appropriate delivery. Pumps were re-tested for accuracy and passed. There was no evidence for dosing or delivery problems,

### Discussion

The study's primary outcome was the apparent safety of PSMA-targeting with dTc. This was not a given. PSMA is expressed in kidney proximal tubule and on type II astrocytes in brain and other sites [22, 23]. In prior dTc trials, serious on-target/off-tumor toxicities could be discerned even by simple infusion with 1st generation (zeta-only) constructs [24] that could be lethal in engraftment settings with 2^{nd} generation dTc (incorporating costimulation) [25], considered the most aggressive exposure [26]. It is therefore reassuring that no CNS, renal or other-site toxicity occurred where anti-PSMA potency was otherwise sufficient to render anti-tumor benefits. Whereas conditioning is itself a serious intervention that can cause deaths [11, 27], and genotoxicity is cited as a hazard of gene therapy [28], these risks, elaborated in the informed consent, were acceptable to these CRPC patients facing early death.

A second objective was to study pharmacokinetics/pharmacodynamics of the infused drugs: dTc and IL2. In the same fashion that area-under-the-curve (AUC) is applied for drug exposure with carboplatin, degree-of-engraftment post-conditioning may be considered as a measure of “drug exposure” with dTc. The benefit of higher, more prolonged effector cell exposures drove recent preferences for engraftment with TIL protocols [11] that informed our study design. Similarly, conditioning was seen to magnify our dTc exposure ≧100-fold (FIG. 1D).

In contrast to carboplatin, however, where AUC is predicted by dose and renal function, this study highlights a vagary of conditioning in that identical dTc doses (Pt1 vs Pt3) achieved 10-fold differences in engraftment (“exposure”), and likewise that similar “drug exposures” occurred with 10-fold different doses (Pt3 vs Pt4) (Table 2A). This makes usual dose-escalation strategies in engraftment settings potentially perilous ventures wherein exposures may be poorly controlled, undermining the concept of managed risk. Even the lowest planned dose (10⁹ cells) could reconstitute to half of total body T cells (e.g., Pt3) that might have yielded a fatal outcome with this self-directed CAR if it acted against normal tissues [25]. This exposure unpredictability could be a further argument for initial safety testing with simple infusions before proceeding to engraftment protocols, particularly where CARs incorporate costimulatory domains that may resist anti-suppressive measures to reverse toxicity [26, 29].

In the case of CD19 CAR in CLL, it was surmised that encounter with large-volume tumor antigen drove their expansions that far exceed even ours [8,9]. Although in vitro studies indicated selective expansions with the 1^{st} generation anti-PSMA CAR dTc on tumor in presence of adequate IL2 [30], our best engraftments clinically had the least evidence for tumor targeting and the lowest IL2, suggesting little if any role for our patients' comparatively smaller-volume prostate cancer target in promoting their dTc expansions. Alternatively, we would propose in our instance that variation in engraftments could derive from different degrees of lymphodepletion, with lesser or greater residual T cells to dilute dTc during recovery/reconstitution, and that may not be predictable on a patient-by-patient basis.

Note: That is, with 10⁹ infused aTc and 10¹⁰ surviving endogenous T cells (a 99% or two-log kill), a reconstitution fraction of −10% (e.g., Pt 2; Table 2A) was likely. For a more effective suppression by conditioning with only 10⁹ surviving endogenous T cells (a 99.9% or three-log kill), a reconstitution fraction of ˜50% might be achieved with the same 10⁹ dose (e.g., Pt 3, Table 2A).

Interestingly, no anti-CAR immune response was detected in any subject despite presence of murine Fv sequences [10], also ruling out immune rejection as source of variability in engraftments. Fv regions are the least immunogenic component of mouse antibodies in humans and vary in their induction of responses [31]. It is possible that conditioning also contributed to this tolerance.

The hope for this method was based on improved effectiveness of TILs in melanoma when engrafted, and on higher engraftments leading to higher response rates [13]. Although we obtained responses in two patients that could support the benefits of engraftment, our results contradicted this latter expectation, responses correlating inversely with engraftment (p=0.06; Table 6B, Response vs Engraftment). Yet high engraftments correlated with low IL2 levels (p<0.01) that were as much as 20-fold under prediction in the critical first week of therapy. The infused activated T cells (aTc) expressing elevated IL2R were postulated to deplete administered IL2 (see above Note 5, with low residual levels insufficient to sustain the activated state of those T cells as needed for tumor cell killing. Once this transformation was applied, responses were seen to correlate directly with resultant IL2 levels (p=0.03) (Table 6C), by which an inverse relation of response to engraftment could now be understandable.

As a therapeutic, IL2 has shown no value in adenocarcinomas outside of renal cell (RCC), with 0 responses among 97 patients with diverse, non-RCC adenocarcinomas, including prostate cancer [32]. By contrast, IL2 is a key component of cellular (TIL) therapies [33], including TIL engraftment protocols [11]. In a murine prostate cancer model, IL2 was likewise of no benefit, but was an essential adjunct to successful adoptive cell therapy [34]. As this model also included conditioning for T cell expansion, the persistent need for IL2 for anti-tumor effect demonstrates that the proliferative, non-activated “recovery” response to homeostatic cytokines (e.g., IL7/IL15) can be separated from an activation/cyto-lytic response that still requires IL2. Our patient data showing absent anti-tumor activity despite vigorous in vivo expansions with low IL2 are consistent with this judgment. Finally, IL2 has been proven essential for dTc to eliminate established solid tumors in animal models, either with IL2 supplied exogenously [35] or by supplementing IL2-secreting CD4+ dTc to high levels in the dose [36] that complement other data in an adoptive cell therapy model [37].

Hence, the value of high IL2 in our responders is conceived as supporting the transferred T cells during their residual period of activation post-infusion. The activation state, as well as the dose size, was previously shown to predict response with TIL [38]. As cited above, melanoma-TIL studies similarly showed higher response rates with higher TIL engraftments post lymphodepletion—but while supported with high dose IL2 (HDI) under the Surgery Branch protocols that is sufficient to saturate IL2R under all conditions of T cell activation and engraftment [13]. With adequate IL2, we may likewise anticipate improved responses with higher engraftments in dTc treatment of prostate cancer.

These results invite comparison with the above-cited CLL study without IL2 [8,9], in which 3 patients were described: 2/3 achieved CR and 1/3 PR, versus 2/5 PR in ours. Factors supporting their deeper responses may include: a. liquid/lymphoid tumor versus solid, b. 3-signal versus 1-signal, and c. dispersed tumor sustaining bulk dTc activation (note 10). Yet, a further anti-CD19 CLL study [39] with 2nd generation dTc with 1/5 PR did not fare objectively better than ours despite having 2 signals and sharing favorable features of CLL tumor type and dispersion, seemingly drawing focus to signal-3 (4-1BB) as a key differentiator. (CD28 Signal 2 in the 4-1BBz dTc is generated gratis via B7 on CLL targets.) Still, having witnessed PRs in prostate cancer with a suboptimal anti-PSMA dTc intervention, it is possible that these qualitative differences may be surmounted by higher dTc exposures/engraftments with adequate IL2.

Recently, Slovin et al [40] reported a series of 7 patients treated on a separate dTc trial targeting PSMA, using a 2^{nd} gen (28z) CAR after prior lymphopenic conditioning with 300 mg/m2 Cy. CAR+ cells persisted in blood for up to 2 wks. There was 0/7 PR but disease stabilization in 2 subjects. A number of differences distinguish their study from ours, including different anti-PSMA Abs in the CARs, our more intensive conditioning regimen, their inclusion of a thymidine kinase safety gene (which could be targeted) and our use of IL2. Our study had robust engraftment, measurable by flow in all patients at a year or more after treatment (or until death) and 2/5 PR. In principle, a difference that could favor the Slovin et al study is presence of a CD28 Signal 2 domain that is absent in our 1^{st} gen construct, yet this benefit did not obviously manifest under their trial. One factor that may mitigate in favor our construct is that our CAR, despite being 1^{st} gen, proliferates on PSMA+ cells with sustained tumor cell killing (Supplemental FIG. 2) [30] rather than succumbing to AICD as commonly seen without CD28 [41, 42]. This proliferation of our 1^{st} gen dTc may be due to specific features of the CAR in our dTc or of the tumor targets (as analogously seen with a 1^{st} gen IL13-zetakine construct in gliomas [43]), and cannot be answered at this time. In any case, the positive results from this clinical study with this agent even at lowest exposures are encouraging for a more complete exploration of its potential as an anti-prostate cancer therapeutic.

In summary, this Phase I trial showed safety of targeting PSMA by designer T cells, quantitated benefit of lymphodepletion to promote dTc engraftment, generated responses in patients with metastatic prostate cancer, and defined systemic IL2 levels as determined by interactions with engrafted T cells as a plausible predictor of clinical response. This report presents a unique example of the pharmacodynamics of drug-drug interactions having a critical impact on the efficacy of their co-application. The potential for IL2 depletion by high engraftments is suggested to limit the gains anticipated with higher dTc exposures, prompting a study redesign with augmented IL2 (note 11; SOM). Where low engraftments of 5-12% with adequate IL2 could induce PSA reductions of 50-70%, high engraftments of up to 60% with enhanced IL2 may provide the 100% PSA reductions and tumor eradications sought with cancer treatment.

### Summary

Patients underwent chemotherapy conditioning, followed by dTc dosing under a Phase I escalation with continuous infusion low dose IL2 (LDI). A target of dTc escalation was to achieve ≧20% engraftment of infused activated T cells.

Six patients enrolled with doses prepared of whom five were treated. Patients received 10⁹ or 10¹⁰ autologous dTc, achieving expansions of 20-560-fold over 2w and engraftments of 5-56%. Pharmacokinetic and pharmacodynamic analyses established the impact of conditioning to promote expansion and engraftment of the infused T cells. Unexpectedly, administered IL2 was depleted up to 20-fold with high activated T cell engraftment in an inverse correlation (p<0.01). Clinically, no anti-PSMA toxicities were noted, and no anti-CAR reactivities were detected. Two-of-five patients achieved partial clinical responses, with PSA declines of 50% and 70% over 1-2+ months and PSA delays of 78 and 150 days, plus a minor response in a third patient. Responses were unrelated to dose size (p=0.6), instead correlating inversely with engraftment (p=0.06) and directly with plasma IL2 (p=0.03), suggesting insufficient IL2 with our LDI protocol to support dTc anti-tumor activity under optimal engraftments.

In conclusion, under a Phase I dose escalation in prostate cancer, a 20% engraftment target was met in three subjects with adequate safety, leading to study conclusion. Clinical responses were obtained, but were suggested to be restrained when activated T cells engrafted to high levels to bind and deplete IL2. This study presents a unique example of how the pharmacodynamics of “drug-drug” interactions may have a critical impact on the efficacy of their co-application.

The foregoing example is also described in Junghans et al. (2016) The Prostate 76:1257.

TABLE 7
SEQUENCE SUMMARY

| SEQ | | |
|---|---|---|
| ID NO: | DESCRIPTION | SEQUENCE |
| 1 | Amino acid sequence | MSPAQFLFLLVLWIQETNGDVVMTQTPLTLSVTIG |
| | of light chain variable | QPASISCKSSQSLLYSNGKTYLNWLLQRPGQSPKR |
| | region of antibody | LIYLVSKLDSGVPDRFTGSGSGTDFTLKISRVEAED |
| | 3D8 | LGVYYCVQGTHFPHTFGGGTKLEIKR |
| 2 | Amino acid sequence | MNFGLSLIFLVLVLKGVQCEVKVVESGGGLVKPG |
| | of heavy chain variable | ASLICLSCAASGFTFSNYGMSWVRQTSDICRLEWVA |
| | region of antibody | SISSGGDSTFYADNVKGRFTISRENAKNTLYLQMS |
| | 3D8 | SLKSEDTALYYCARDDLFNVVGQGTTLTVSS |
| 3 | Amino acid sequence | GKPIPNPLLGLDST |
| | of V5 tag | |
| 4 | Amino acid sequence | KPTTTPAPRPPTPAPTIASQPLSLRPEACRPAAGGA |
| | of CDR hinge region | VHTRCHDFA |
| 5 | Amino acid sequence | LDPKLCYLLDGILFIYGVILTALFLRVICFSRSADAPA |
| | of CD3 zeta signaling | YQQGQNQLYNELNLGRREEYDVLDKRRGRDPEM |
| | region | GGKPRRKNPQEGLYNELQICDICMAEAYSEIGMKGE |
| | | RRRGKGHDGLYQGLSTATKDTYDALHMQALPPR |
| 6 | Amino acid sequence | SSNEATNITPKHNMKAFLDELKAENIKKFLYNFTQI |
| | of human PSMA | PHLAGTEQNFQLAKQIQSQWKEFGLDSVELAHYD |
| | | VLLSYPNKTHPNYISIINEDGNEIFNTSLFEPPPPGY |
| | | ENVSDIVPPFSAFSPQGMPEGDLVYVNYARTEDFF |
| | | KLERDMKINCSGKIVIARYGKVFRGNKVKNAQLA |
| | | GAKGVILYSDPADYFAPGVKSYPDGWNLPGGGVQ |
| | | RGNILNLNGAGDPLTPGYPANEYAYRRGIAEAVGL |
| | | PSIPVHPIGYYDAQICLLEICMGGSAPPDSSWRGSLK |
| | | VPYNVGPGFTGNFSTQKVKMHIHSTNEVTRIYNVI |
| | | GTLRGAVEPDRYVILGGHRDSWVFGGIDPQSGAA |
| | | VVHEIVRSFGTLICKEGWRPRRTILFASWDAEEFGL |
| | | LGSTEWAEENSRLLQERGVAYINADSSIEGNYTLR |
| | | VDCTPLMYSLVHNLTKELKSPDEGFEGKSLYESW |
| | | TICKSPSPEFSGMPRISICLGSGNDFEVFFQRLGIASG |
| | | RARYTICNWETNICFSGYPLYHSVYETYELVEKFYD |
| | | PMFKYHLTVAQVRGGMVFELANSIVLPFDCRDYA |
| | | VVLRKYADICIYSISMICHPQEMKTYSVSFDSLFSAV |
| | | KNFTEIASKFSERLQDFDKSNPIVLRMMNDQLMFL |
| | | ERAFIDPLGLPDRPFYRHVIYAPSSHNKYAGESFPGI |
| | | YDALFDIESKVDPSKAWGEVKRQIYVAAFTVQAA |
| | | AETLSEVA |
| 7 | Mature amino acid | APTSSSTICKTQLQLEHLLLDLQMILNGINNYKNPK |
| | sequence of human | LTRMLTFICFYMPICKATELICHLQCLEEELKPLEEVL |
| | IL2 | NLAQSKNFHLRPRDLISNINVIVLELKGSETTFMCE |
| | | YADETATIVEFLNRWITFCQSIISTLT |
| 8 | Amino acid sequence | PTSSSTICKTQLQLEHLLLDLQMILNGINNYKNPICLT |
| | of des-alanyls-1, serine | RMLTFICFYMPICKATELICHLQCLEEELKPLEEVLNL |
| | 125 human IL2 | AQSKNFHLRPRDLISNINVIVLELKGSETTFMCEYA |
| | | DETATIVEFLNRWITFSQSIISTLT |
| 9 | Amino acid sequence | IPNPLLGLDST |
| | of truncated V5 tag | |
| 10 | Amino acid sequence | MEWSWVFLFFLSVTTGVHS |
| | of signal peptide | |
| 11 | Amino acid sequence | LDPK |
| | of CD3 zeta | |
| | extracellular domain | |
| 12 | Amino acid sequence | LCYLLDGILFIYGVILTALFL |
| | of CD3 zeta | |
| | transmembrane domain | |
| 13 | Amino acid sequence | LDPKLCYLLDGILFIYGVILTALFLRVK |
| | of CD3 zeta | |
| | transmembrane domain | |
| | amino acid sequence | |
| 14 | Amino acid sequence | RVKFSRSADAPAYQQGQNQLYNELNLGRREEYDV |
| | of CD3 zeta | LDKRRGRDPEMGGICPRRICNPQEGLYNELQICDICM |
| | intracellular domain | AEAYSEIGMKGERRRGKGHDGLYQGLSTATKDTY |
| | | DALHMQALPPR |
| 15 | Amino acid sequence | RSKRSRLLHSDYMNMTPRRPGPTRKHYQPYAPPR |
| | of CD28 intracellular | DFAAYRS |
| | domain | |
| 16 | Amino acid sequence | KIEVMYPPPYLDNEKSNGTIIHVKGICHLCPSPLFPG |
| | of CD28 signaling | PSKPFWVLVVVGGVLACYSLLVTVAFTIFWVRSICR |
| | region | SRLLHSDYMNMTPRRPGPTRICHYQPYAPPRDFAA |
| | | YRS |
| 17 | Amino acid sequence | KIEVMYPPPYLDNEKSNGTIIHVKGICHLCPSPLFPG |
| | of CD28 extracellular | PSKP |
| | domain | |
| 18 | Amino acid sequence | FWVLVVVGGVLACYSLLVTVAFIIFWV |
| | of CD28 | |
| | transmembrane domain | |
| 19 | Amino acid sequence | RSKRSRLLHSDYMNMTPRRPGPTRICHYQPYAPPR |
| | of CD28 intracellular | DFAAYRS |
| | domain | |
| 20 | Amino acid sequence | KRGRKKLLYIFKQPFMRPVQTTQEEDGCSCRFPEE |
| | of 4-1BB intracellular | EEGGCEL |
| | domain | |
| 21 | Amino acid sequence | GGSGSGGSGSGGSGS |
| | of linker | |
| 22 | Amino acid sequence | (Gly₄Ser)₃ |
| | of linker | |
| 23 | Amino acid sequence | (Gly₄Ser)₄ |
| | of linker | |
| 24 | Amino acid sequence | (Gly₄Ser)₆ |
| | of linker | |
| 25 | Amino acid sequence | (Gly₄Ser)₉ |
| | of linker | |
| 26 | Amino acid sequence | (Gly₄Ser)₁₂ |
| | of linker | |
| 27 | Amino acid sequence | (Gly₄Ser)₁₅ |
| | of linker | |
| 28 | Amino acid sequence | (Gly₄Ser)₃₀ |
| | of linker | |
| 29 | Amino acid sequence | (Gly₄Ser)₄₅ |
| | of linker | |
| 30 | Amino acid sequence | (Gly₄Ser)₆₀ |
| | of linker | |
| 31 | Amino acid sequence | (Gly₄Ser)ₙ, where n is a positive |
| | of linker | integer equal to or greater than 1. |
| 32 | Nucleic acid sequence | AGGCTGAGGATTTGGGAGTT |
| | of anti-PSMA primer | |
| 33 | Nucleic acid sequence | AGACGCTCCAGGCTTCACTA |
| | of anti-PSMA primer | |
| | of anti-CEA primer | |
| 34 | Nucleic acid sequence | GCAAGCATTACCAGCCCTAT |
| | of anti-CEA primer | |
| 35 | Nucleic acid sequence | GTTCTGGCCCTGCTGGTA |
| | of anti-CEA primer | |
| 36 | Nucleic acid sequence | ACCATGCTTTTCAGCTCTGG |
| | of albumin primer | |
| 37 | Nucleic acid sequence | TCTGCATGGAAGGTGAATGT |
| | of albumin primer | |

### ABBREVIATIONS

ADT androgen deprivation therapy
aTc activated T cell
CAR chimeric antigen receptor
CRPC castrate resistant prostate cancer
civi continuous intravenous infusion
Cy cyclophosphamide
dTc designer T cell
Flu fludarabine
IL2/7/15 interleukin 2/7/15
LDI low dose IL2
MDI medium dose IL2
PSA prostate specific antigen
PSMA prostate specific membrane antigen
SOM supplemental online material
TIL tumor-infiltrating lymphocyte

### REFERENCES

1. Siegel R, Naishadham D, Jemal A. Cancer statistics, 2012. CA Cancer J Clin 2012; 62:10-29.
2. De Bono J S, Logothetis C J, Molina A et al. Abiraterone and increased survival in metastatic prostate cancer. N Engl J Med 2011; 364:1995-05.
3. Scher H, Fazazi K, Saad F. Effect of MDV3100, an androgen receptor signaling inhibitor (ARSI), on overall survival in patients with prostate cancer post docetaxel: Results from the phase III AFFIRM study. J Clin Oncol 2012.
4. Berthold D R, Pond G R, Soban F, et al. Docetaxel plus prednisone or mitoxantrone plus prednisone for advanced prostate cancer: updated survival in the TAX 327 study. J Clin Oncol. 2008; 26:242-5.
5. De Bono J S, Oudard S, Ozguroglu M, et al. Prednisone plus cabazitaxel or mitoxantrone for metastatic castration-resistant prostate cancer progressing after docetaxel treatment: a randomised open-label trial. Lancet 2005; 376:1147-54.
6. Kantoff P W, Higano C S, Shore N D, et al. Sipuleucel-T immunotherapy for castration-resistant prostate cancer. N Engl J Med 2008; 363:411-22.
7. Ma Q, Gonzalo-Daganzo R M, Junghans R P. Genetically engineered T cells as adoptive immunotherapy of cancer. Cancer Chemother Biol Response Modif 2002; 20:315-41.
8. Porter D L, Levine B L, Kalos M, et al. Chimeric antigen receptor-modified cells in chronic lymphoid leukemia. N Engl J Med 2011; 365:725-33.
9. Kalos M, Levine B L, Porter D L, et al. T cells with chimeric antigen receptors have potent antitumor effects and can establish memory in patients with advanced leukemia. Sci Transl Med. 2011; 3:95ra73.
10. Ma, Q, Safar M, Holmes E, et al. Anti-prostate specific membrane antigen designer T cells for prostate cancer therapy. Prostate 2004; 61:12-25.
11. Dudley M E, Wunderlich J R, Yang J C et al. Adoptive cell transfer therapy following non-myeloablative but lymphodepleting chemotherapy for the treatment of patients with refractory metastatic melanoma. J Clin Oncol 2005; 23:2346-57.
12. Bracci L, Moschella F, Sestili P, et al. Cyclophosphamide enhances the antitumor efficacy of adoptively transferred immune cells through the induction of cytokine expression, B-cell and T-cell homeostatic proliferation, and specific tumor infiltration. Clin Cancer Res 2007; 13: 644-53.
13. Dudley M E, Yang J C, Sherry R, et al. Adoptive cell therapy for patients with metastatic melanoma: evaluation of intensive myeloablative chemoradiation preparative regimens. J Clin Oncol 2008; 26: 5233-9.
14. Konrad M W, Hemstreet G, Hersh E M, et al. Pharmacokinetics of recombinant interleukin 2 in humans. Cancer Res 1990; 50:2009-17.
15. Kochenderfer J N, Dudley M E, Feldman S A, Wilson W H, Spaner D E, et al. B-cell depletion and remissions of malignancy along with cytokine-associated toxicity in a clinical trial of anti-CD19 chimeric-antigen-receptor-transduced T cells. Blood. 2012; 119:2709-20.
16. Brentjens R J, Davila M L, Riviere I, Park J, Wang X, et al. CD19-targeted T cells rapidly induce molecular remissions in adults with chemotherapy-refractory acute lymphoblastic leukemia. Sci Transl Med. 2013; 5:177ra38.
17. Antonarakis E S, Zahurak M L, Lin J, et al. Changes in PSA kinetics predict metastasis-free survival in men with PSA-recurrent prostate cancer treated with nonhormonal agents: combined analysis of 4 phase II trials. Cancer 2001; 118:1533-42.
18. Hussain M, Goldman B, Tangen C, et al. Prostate-Specific Antigen progression predicts overall survival in patients with metastatic prostate cancer: Data from Southwest Oncology Group Trials 9346 (Intergroup study 0162) and 9916. J of Clin Oncol 2009; 27:2450-6.
19. Armstrong A J, Eisenberger M A, Halabi S, et al. Biomarkers in the management and treatment of men with metastatic castration-resistant prostate cancer. Eur Urol. 2011; 61:549-59.
20. Madan R A, Bilusic M, Heery C, et al. Clinical evaluation of TRICOM vector therapeutic cancer vaccines. Semin Oncol. 2012; 39:296-304.
21. Stein W D, Gulley J L, Schlom J, et al. Tumor regression and growth rates determined in five intramural NCI prostate cancer trials: the growth rate constant as an indicator of therapeutic efficacy. Clin Cancer Res. 2012; 17:907-17.
22. Kinoshita Y, Kuratsukuri K, Landas S, et al. Expression of prostate-specific membrane antigen in normal and malignant human tissues. World J Surg. 2006; 30:628-36.
23. Sacha P, Zamecnik J, Barinka C, et al. Expression of glutamate carboxypeptidase II in human brain. Neuroscience 2007; 144:1361-72.
24. Lamers C H, Sleijfer S, Vulto A G, et al. Treatment of metastatic renal cell carcinoma with autologous T-lymphocytes genetically retargeted against carbonic anhydrase IX: first clinical experience. J Clin Oncol. 2006; 24:e20-2.
25. Morgan R A, Yang J C, Kitano M, et al. Case report of a serious adverse event following the administration of T cells transduced with a chimeric antigen receptor recognizing ERBB2. Mol Ther. 2010; 18:843-51.
26. Junghans R P. Strategy Escalation: An emerging paradigm for safe clinical development of T cell gene therapies. J Transl Med. 2010; 8:55.
27. Brentjens R, Yeh R, Bernal Y, et al., Treatment of chronic lymphocytic leukemia with genetically targeted autologous T cells: case report of an unforeseen adverse event in a phase I clinical trial. Mol Ther. 2010; 18:666-8.
28. FDA. FDA Guidance for Industry, Gene therapy clinical trials—observing participants for delayed adverse events. Food & Drug Administration, 2005.
29. Junghans RP. Is it safer CARs that we need, or safer rules of the road? Mol Ther. 2010; 10:1742-3.
30. Ma Q, Gomes E, Bais A J, Junghans R P. Advanced generation anti-prostate specific membrane antigen (PSMA) “designer T cells” for prostate cancer immunotherapy. New England Immunology Conference, Woods Hole, Mass., 2009.
31. Scheinberg D B, Rosenblat T, Jurcic J G, et al. Antibody-based immunotherapies for cancer. (Chap. 25) In Chabner B A, Longo D L (eds) Cancer Chemotherapy and Biotherapy, 5th Edition. Philadelphia: Lippincott, 2010: 465-94.
32. Rosenberg S A, Lotze M T, Yang J C, et al. Experience with the use of high-dose interleukin-2 in the treatment of 652 cancer patients. Ann Surg. 1989; 210:474-84; discussion 484-5.
33. Rosenberg S A, Yannelli J R, Yang J C, et al. Treatment of patients with metastatic melanoma with autologous tumor-infiltrating lymphocytes and interleukin 2. J Natl Cancer Inst. 1994; 86:115966.
34. Yi H, Yu X, Guo C, et al. Adoptive cell therapy of prostate cancer using female mice-derived T cells that react with prostate antigens. Cancer Immunol Immunother. 2011; 60:349-60.
35. Lo A S, Ma Q, Liu D L, et al., Anti-GD3 chimeric sFv-CD28/T-cell receptor zeta designer T cells for treatment of metastatic melanoma and other neuroectodermal tumors. Clin Cancer Res. 2010; 16:2769-80.
36. Moeller M, Haynes N M, Kershaw M H, et al. Adoptive transfer of gene-engineered CD4+ helper T cells induces potent primary and secondary tumor rejection. Blood 2005; 106:2995-03.
37. Antony P A, Piccirillo C A, Akpinarli A, et al. CD8+ T cell immunity against a tumor/self-antigen is augmented by CD4+ T helper cells and hindered by naturally occurring T regulatory cells. J Immunol. 2005; 174:2591-601.
38. Schwartzentruber D J, Hom S S, Dadmarz R, et al. In vitro predictors of therapeutic response in melanoma patients receiving tumor-infiltrating lymphocytes and interleukin-2. J Clin Oncol. 1994; 12:1475-83.
39. Brentjens R J, Rivière I, Park J H, Davila M L, Wang X, Stefanski J, Taylor C, Yeh R, Bartido S, Borquez-Ojeda O, Olszewska M, Bernal Y, Pegram H, Przybylowski M, Hollyman D, Usachenko Y, Pirraglia D, Hosey J, Santos E, Halton E, Maslak P, Scheinberg D, Jurcic J, Heaney M, Heller G, Frattini M, Sadelain M. Safety and persistence of adoptively transferred autologous CD19-targeted T cells in patients with relapsed or chemotherapy refractory B-cell leukemias. Blood. 2011; 118:4817-28.
40. Slovin S F, Wang X, Hullings M, Arauz G, Bartido S, Lewis J S, Schoder H, Zanzonico P, Scher H I, Sadelain M, Riviere I. Chimeric antigen receptor (CAR⁺) modified T cells targeting prostate-specific membrane antigen (PSMA) in patients (pts) with castrate metastatic prostate cancer (CMPC). J Clin Oncol 31, 2013 (suppl 6; abstr 72)
41. Beecham E J, Ma Q Z, Ripley R, Junghans R P. Coupling of CD28 co-stimulation to IgTCR molecules: Dynamics of T cell proliferation and death. J Immunother 2000; 23:631-42.
42. Emtage P C R, Lo A S Y, Gomes E M, Liu D L, Gonzalo-Daganzo R, Junghans R P. 2nd generation anti-CEA designer T cells resist activation-induced cell death, proliferate on tumor contact, secrete cytokines and exhibit superior anti-tumor activity in vivo: a preclinical evaluation. Clin Cancer Res 2008; 14:8112-22.
43. Kahlon K S, Brown C, Cooper L J, Raubitschek A, Forman S J, Jensen M C. Specific recognition and killing of glioblastoma multiforme by interleukin 13-zetakine redirected cytolytic T cells. Cancer Res. 2004; 64:9160-6.

### ADDITIONAL LITERATURE

Alvarez-Vallina L, Hawkins R E. Antigen-specific targeting of CD28-mediated T cell co-stimulation using chimeric single-chain antibody variable fragment-CD28 receptors. Eur J Immunol. 1996; 26:2304-9.
Baccala A, Sercia L, Li J, Heston W, Zhou M. Expression of prostate-specific membrane antigen in tumor-associated neovasculature of renal neoplasms. Urology. 2007; 70:385-90.
Beecham E J, Ma Q Z, Ripley R, Junghans R P. Coupling of CD28 co-stimulation to IgTCR molecules: Dynamics of T cell proliferation and death. J Immunother 2000; 23:631-42.
Benarroch E E. Neuron-astrocyte interactions: partnership for normal function and disease in the central nervous system. Mayo Clin Proc. 2005; 80:1326-38.
Bracci L, Moschella F, Sestili P, La Sorsa V, Valentini M, Canini I, Baccarini S, Maccari S, Ramoni C, Belardelli F, Proietti E. Cyclophosphamide enhances the antitumor efficacy of adoptively transferred immune cells through the induction of cytokine expression, B-cell and T-cell homeostatic proliferation, and specific tumor infiltration. Clin Cancer Res. 2007; 13 (2Pt 1):644-53.
Brentjens R J, Riviere I, Park J H, Davila M L, Wang X, Stefanski J, Taylor C, Yeh R, Bartido S, Borquez-Ojeda O, Olszewska M, Bernal Y, Pegram H, Przybylowski M, Hollyman D, Usachenko Y, Pirraglia D, Hosey J, Santos E, Halton E, Maslak P, Scheinberg D, Jurcic J, Heaney M, Heller G, Frattini M, Sadelain M. Safety and persistence of adoptively transferred autologous CD19-targeted T cells in patients with relapsed or chemotherapy refractory B-cell leukemias. Blood. 2011; 118:4817-28.
Chlebowski R T, Hestorff R, Sardoff L, et al. Cyclophosphamide (NSC 26271) versus the combination of adriamycin (NSC 123127), 5-fluorouracil (NSC 19893), and cyclophosphamide in the treatment of metastatic prostatic cancer: a randomized trial. Cancer 1978; 42:2546-52.
Dudley M E, Wunderlich J R, Yang J C et al. Adoptive cell transfer therapy following non-myeloablative but lymphodepleting chemotherapy for the treatment of patients with refractory metastatic melanoma. J Clin Oncol 2005; 23:2346-57.
Emtage P C R, Lo A S Y, Gomes E M, Liu D L, Gonzalo-Daganzo R, Junghans R P. 2nd generation anti-CEA designer T cells resist activation-induced cell death, proliferate on tumor contact, secrete cytokines and exhibit superior anti-tumor activity in vivo: a preclinical evaluation. Clin Cancer Res 2008; 14:8112-22.
Hank J A, Surfus J, Gan J, Albertini M, Lindstrom M, Schiller J H, Hotton K M, Khorsand M, Sondel P M. Distinct clinical and laboratory activity of two recombinant interleukin-2 preparations. Clin Cancer Res. 1999; 5:281-9.
Jacques Y, Le Mauff B, Boeffard F, Godard A, Soulillou J P. A soluble interleukin 2 receptor produced by a normal alloreactive human T cell clone binds interleukin 2 with low affinity. J Immunol. 1987; 139:230816.
Junghans R P, Safar M, Huberman M S, Ma Q, Ripley R, Leung S, Beecham E J. Preclinical and phase I data of anti-CEA “designer T cell” therapy for cancer: A new immunotherapeutic modality. Proc Am Soc Clin Oncol 2001: A1063.
Konrad M W, Hemstreet G, Hersh E M, Mansell P W, Mertelsmann R, Kolitz J E, Bradley E C. Pharmacokinetics of recombinant interleukin 2 in humans. Cancer Res 1990; 50:2009-17.
Lo A S Y, Ma Q, Liu D L, Junghans R P. Anti-GD3 chimeric sFv-CD28/T cell receptor zeta designer T cells for treatment of metastatic melanoma and other neuroectodermal tumors. Clin Cancer Res 2010; 16:2769-80.
Ma Q, Gomes E, Bais A J, Junghans R P. Advanced generation anti-prostate specific membrane antigen (PSMA) “designer T cells” for prostate cancer immunotherapy. New England Immunology Conference, Woods Hole, Mass., 2010.
McDermott D F, Regan M M, Clark J I, Flaherty L E, Weiss G R, Logan T F, Kirkwood J M, Gordon M S, Sosman J A, Ernstoff M S, Tretter C P, Urba W J, Smith J W, Margolin K A, Mier J W, Gollob J A, Dutcher J P, Atkins M B. Randomized phase III trial of high-dose interleukin-2 versus subcutaneous interleukin-2 and interferon in patients with metastatic renal cell carcinoma. J Clin Oncol. 2005; 23:133-41. Erratum in: J Clin Oncol. 2005; 23:2877.
Muss H B, Howard V, Richards F, et al. Cyclophosphamide versus cyclophosphamide, methotrexate, and 5-fluorouracil in advanced prostatic cancer: a randomized trial. Cancer 1981; 47:1949-53 Rosenberg S A, Yannelli J R, Yang J C et al. Treatment of patients with metastatic melanoma with autologous tumor-infiltrating lymphocytes and interleukin 2. J Natl Cancer Inst 1994; 86:1159-66.
Saxman S, Ansari R, Drasga R, et al. Phase III trial of cyclophosphamide versus cyclophosphamide, doxorubicin, and methotrexate in hormone-refractory prostatic cancer. A Hoosier Oncology Group study. Cancer 1992; 70:2488-92.
Schwartzentruber D J, Horn S S, Dadmarz R, White D E, Yannelli J R, Steinberg S M, Rosenberg S A, Topalian S L. In vitro predictors of therapeutic response in melanoma patients receiving tumor-infiltrating lymphocytes and interleukin-2. J Clin Oncol. 1994; 12:1475-83.
Beaudoin E L, Bais A J, Junghans R P. Sorting vector producer cells for high transgene expression increases retroviral titer. J Virol Meth 2008; 148:253-9.
Ma Q Z, Safar M, Holmes E, Wang Y W, Boynton A L, Junghans R P. Anti-prostate specific membrane antigen designer T cells for prostate cancer therapy. Prostate 2004a: 61:12-25.
Ma Q Z, DeMarte L, Wang Y W, Stanners C P, Junghans R P. Carcinoembryonic antigen-immunoglobulin Fc fusion protein (CEA-Fc) for identification and activation of anti-CEA chimeric immune receptor modified T cells: representative of a new class of Ig fusion proteins. Cancer Gene Ther 2004b: 11:297-306.

1. A method of treating prostate cancer in a human subject in need thereof, comprising administering to the subject a population of cells expressing a chimeric antigen receptor (CAR) which specifically binds prostate specific membrane antigen (PSMA) and administering interleukin-2 (IL2), thereby treating prostate cancer in the human subject, wherein the IL2 is administered to the human subject by continuous intravenous infusion at a dose of about 75000 IU/kg/d and is administered after administration of the population of cells expressing the CAR.

2. The method of claim 1, further comprising administering cyclophosphamide and/or fludarabine to the human subject.

3. The method of claim 1, wherein the IL2 is administered to the subject for about 28 days by continuous intravenous infusion.

4. The method claim 1, wherein the CAR comprises a PSMA binding region of an anti-PSMA antibody and a CD3 zeta signaling region of a T cell receptor.

5. The method of claim 4, wherein the anti-PSMA antibody is 3D8, or an antigen binding fragment thereof.

6. A method of treating a human subject having prostate cancer, said method comprising administering a population of cells expressing an anti-PSMA CAR to the human subject and administering IL2 to the human subject, wherein the IL2 is administered intravenously to the human subject at a dose of 100 kIU/kg/8 h or more by bolus infusion and is administered after administration of the population of cells expressing the anti-PSMA CAR, and wherein the anti-PSMA CAR comprises an anti-PSMA scFv, a transmembrane domain, and a CD3 zeta signaling region.

7.-12. (canceled)

13. The method of claim 1, wherein the population of cells comprises T-cells obtained from the subject.

14. A method of treating prostate cancer in a subject infused with a population of cells expressing an anti-PSMA CAR, said method comprising administering IL2 to the subject according to a dosing schedule such that an IL2 plasma level of greater than 500 pg/ml is maintained in the subject for at least a week following administration of the population of cells to the subject, wherein the anti-PSMA CAR comprises an extracellular region comprising an anti-PSMA scFv, a transmembrane domain, and a CD3 zeta signaling region.

15. The method of claim 14, wherein the IL2 plasma level is maintained for one to two weeks following administration of the population of cells to the subject.

16. (canceled)

17. The method of claim 14, wherein the IL2 plasma level is maintained for a month following administration of the population of cells to the subject.

18. (canceled)

19. The method of claim 14, wherein the subject has an activated cell engraftment of at least 10%.

20. The method of claim 14, wherein the subject has an activated cell engraftment of at least 50%.

21. A method of treating cancer in a subject who has been infused with a population of cells expressing a CAR which is specific for a cancer antigen, said method comprising administering IL2 to the subject according to a dosing schedule such that an IL2 plasma level of greater than 500 pg/ml is maintained in the subject for at least a week following administration of the population of cells to the subject, wherein the subject has received lymphodepletion therapy prior to administration of the population of cells to the subject.

22. A method of treating cancer in a subject, said method comprising administering a population of cells expressing a CAR which is specific for a cancer antigen to the subject having cancer and subsequently administering IL2 to the subject either by bolus infusion comprising administering a dose of IL2 of 100 kIU/kg/8 h or more, or by continuous infusion comprising administering 25000 IU/kg/d to 300000 IU/kg/d of IL2 to the subject, wherein the subject has received lymphodepletion therapy prior to administration of the population of cells to the subject.

23. (canceled)

24. The method of claim 21, wherein the cancer is selected from the group consisting of colon cancer, breast cancer, brain cancer, lung cancer, ovarian cancer, head and neck cancer, bladder cancer, melanoma, colorectal cancer, and pancreatic cancer.

25. The method of claim 21, wherein the cancer antigen is selected from the group consisting of carcino-embryonic antigen (CEA), CD19, GM2, GD2, sialyl Tn (STn), HER2, EGFR, GD3, IL13R, MUC-1, and EGFRvIII.

26. The method of claim 1, wherein the IL2 is aldesleukin (Proleukin).

27. The method of claim 4, wherein the anti-PSMA scFv comprises a light chain variable region comprising the amino acid sequence as set forth in SEQ ID NO: 1, and comprising a heavy chain variable region comprising the amino acid sequence as set forth in SEQ ID NO: 2.

28. The method of claim 4, wherein the anti-PSMA CAR comprises a CD8 hinge region.

29.-30. (canceled)

31. The method of claim 1, wherein the prostate cancer is associated with PSMA expression.

32. The method of claim 1, wherein the prostate cancer is metastatic prostate cancer, recurrent prostate cancer, or hormone-refractory prostate cancer.

33. (canceled)


SEQUENCE LISTING (37 sequences)

SEQ ID NO: 1 - protein, 132 aa, Artificial Sequence
  Synthetic Amino acid sequence of light chain variable region of antibody 3D8
MSPAQFLFLLVLWIQETNGDVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPHTFGGGTKLEIKR

SEQ ID NO: 2 - protein, 133 aa, Artificial Sequence
  Synthetic Amino acid sequence of heavy chain variable region of antibody 3D8
MNFGLSLIFLVLVLKGVQCEVKVVESGGGLVKPGASLKLSCAASGFTFSNYGMSWVRQTSDKRLEWVASISSGGDSTFYADNVKGRFTISRENAKNTLYLQMSSLKSEDTALYYCARDDLFNWGQGTTLTVSS

SEQ ID NO: 3 - protein, 14 aa, Artificial Sequence
  Synthetic Amino acid sequence of V5 tag
GKPIPNPLLGLDST

SEQ ID NO: 4 - protein, 45 aa, Artificial Sequence
  Synthetic Amino acid sequence of CD8 hinge region
KPTTTPAPRPPTPAPTIASQPLSLRPEACRPAAGGAVHTRGLDFA

SEQ ID NO: 5 - protein, 137 aa, Artificial Sequence
  Synthetic Amino acid sequence of CD3 zeta signaling region
LDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 706 aa, Homo sapiens
  misc_feature: Amino acid sequence of human PSMA
SSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 7 - protein, 133 aa, Homo sapiens [sequence omitted]

SEQ ID NO: 8 - protein, 132 aa, Artificial Sequence
  Synthetic Amino acid sequence of des-alanyls-1, serine 125 human IL2
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - protein, 11 aa, Artificial Sequence
  Synthetic Amino acid sequence of truncated V5 tag
IPNPLLGLDST

SEQ ID NO: 10 - protein, 19 aa, Artificial Sequence
  Synthetic Amino acid sequence of signal peptide
MEWSWVFLFFLSVTTGVHS

SEQ ID NO: 11 - protein, 4 aa, Artificial Sequence
  Synthetic Amino acid sequence of CD3 zeta extracellular domain
LDPK

SEQ ID NO: 12 - protein, 21 aa, Artificial Sequence
  Synthetic Amino acid sequence of CD3 zeta transmembrane domain
LCYLLDGILFIYGVILTALFL

SEQ ID NO: 13 - protein, 28 aa, Artificial Sequence
  Synthetic Amino acid sequence of CD3 zeta transmembrane domain amino acid sequence
LDPKLCYLLDGILFIYGVILTALFLRVK

SEQ ID NO: 14 - protein, 112 aa, Artificial Sequence
  Synthetic Amino acid sequence of CD3 zeta intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 41 aa, Artificial Sequence
  Synthetic Amino acid sequence of CD28 intracellular domain
RSKRSRLLHSDYMNMTPRRPGPTRKHYQPYAPPRDFAAYRS

SEQ ID NO: 16 - protein, 108 aa, Artificial Sequence
  Synthetic Amino acid sequence of CD28 signaling region
KIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 17 - protein, 40 aa, Artificial Sequence
  Synthetic Amino acid sequence of CD28 extracellular domain
KIEVMYPPPYLDNEKSNGTIIHVKGKHLCPSPLFPGPSKP

SEQ ID NO: 18 - protein, 27 aa, Artificial Sequence
  Synthetic Amino acid sequence of CD28 transmembrane domain
FWVLVVVGGVLACYSLLVTVAFIIFWV

SEQ ID NO: 19 - protein, 41 aa, Artificial Sequence; identical to SEQ ID NO: 15
  Synthetic Amino acid sequence of CD28 intracellular domain

SEQ ID NO: 20 - protein, 42 aa, Artificial Sequence
  Synthetic Amino acid sequence of 4-1BB intracellular domain
KRGRKKLLYIFKQPFMRPVQTTQEEDGCSCRFPEEEEGGCEL

SEQ ID NO: 21 - protein, 15 aa, Artificial Sequence
  Synthetic Amino acid sequence of linker
GGSGSGGSGSGGSGS

SEQ ID NO: 22 - protein, 15 aa, Artificial Sequence
  Synthetic Amino acid sequence of linker
GGGGSGGGGSGGGGS

SEQ ID NO: 23 - protein, 20 aa, Artificial Sequence
  Synthetic Amino acid sequence of linker
GGGGSGGGGSGGGGSGGGGS

SEQ ID NO: 24 - protein, 30 aa, Artificial Sequence
  Synthetic Amino acid sequence of linker
GGGGSGGGGSGGGGSGGGGSGGGGSGGGGS

SEQ ID NO: 25 - protein, 45 aa, Artificial Sequence
  Synthetic Amino acid sequence of linker
GGGGSGGGGSGGGGSGGGGSGGGGSGGGGSGGGGSGGGGSGGGGS

SEQ ID NO: 26 - protein, 60 aa, Artificial Sequence
  Synthetic Amino acid sequence of linker
GGGGSGGGGSGGGGSGGGGSGGGGSGGGGSGGGGSGGGGSGGGGSGGGGSGGGGSGGGGS

SEQ ID NO: 27 - protein, 75 aa, Artificial Sequence
  Synthetic Amino acid sequence of linker
GGGGSGGGGSGGGGSGGGGSGGGGSGGGGSGGGGSGGGGSGGGGSGGGGSGGGGSGGGGSGGGGSGGGGSGGGGS

SEQ ID NO: 28 - protein, 150 aa, Artificial Sequence
  Synthetic Amino acid sequence of linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 29 - protein, 225 aa, Artificial Sequence
  Synthetic Amino acid sequence of linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 30 - protein, 300 aa, Artificial Sequence
  Synthetic Amino acid sequence of linker
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 31 - protein, 5 aa, Artificial Sequence
  Synthetic Amino acid sequence of linker
  misc_feature: Gly4Ser repeats n times, where n is a positive integer equal to or greater than 1
GGGGS

SEQ ID NO: 32 - DNA, 20 bp, Artificial Sequence
  Synthetic Nucleic acid sequence of anti-PSMA primer
aggctgaggatttgggagtt

SEQ ID NO: 33 - DNA, 20 bp, Artificial Sequence
  Synthetic Nucleic acid sequence of anti-PSMA primer
agacgctccaggcttcacta

SEQ ID NO: 34 - DNA, 20 bp, Artificial Sequence
  Synthetic Nucleic acid sequence of anti-CEA primer
gcaagcattaccagccctat

SEQ ID NO: 35 - DNA, 18 bp, Artificial Sequence
  Synthetic Nucleic acid sequence of anti-CEA primer
gttctggccctgctggta

SEQ ID NO: 36 - DNA, 20 bp, Artificial Sequence
  Synthetic Nucleic acid sequence of albumin primer
accatgcttttcagctctgg

SEQ ID NO: 37 - DNA, 20 bp, Artificial Sequence
  Synthetic Nucleic acid sequence of albumin primer
tctgcatggaaggtgaatgt